(19) United States
(12) Patent Application Publication
(10) Pub. No.: US 2025/0043067 A1
(43) Pub. Date: Feb. 6, 2025

(54) TETHERED ALKYLIDENES FROM ORGANOAZIDES FOR CYCLIC POLYMER SYNTHESIS

(71) Applicant: UNIVERSITY OF FLORIDA RESEARCH FOUNDATION, INCORPORATED, Gainesville, FL (US)

(72) Inventors: Adam S. Veige, Gainesville, FL (US); Rinku Yadav, Gainesville, FL (US)

(21) Appl. No.: 18/769,540

(22) Filed: Jul. 11, 2024

Related U.S. Application Data
(63) Continuation of application No. PCT/US22/53852, filed on Dec. 22, 2022.
(60) Provisional application No. 63/298,843, filed on Jan. 12, 2022.

(51) Int. Cl. C08G 61/08

(52) U.S. Cl. CPC C08G 61/08; C08G 2261/373; C08G 2261/418

(57) ABSTRACT

Provided herein are catalysts for polymerization of linear alkynes to cyclic poly(alkynes), and methods of making and using same. For example, provided herein are compounds of formula (I), or dimers thereof:

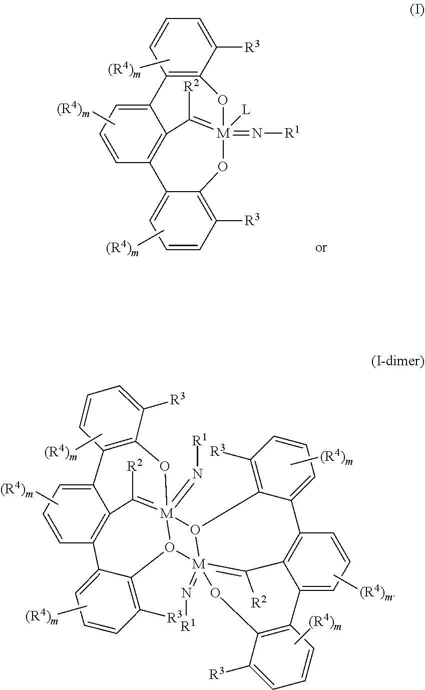

### CROSS-REFERENCE TO RELATED APPLICATIONS

This application is a Continuation-In-Part of International Application No. PCT/US22/53852, filed Dec. 22, 2022, which claims the benefit under 35 USC 119(e) of U.S. Provisional Patent Application No. 63/298,843, filed Jan. 12, 2022, the entire disclosures of which are incorporated herein by reference.

### STATEMENT OF GOVERNMENT SUPPORT

This invention was made with government support under Grant No. 1856674, awarded by the National Science Foundation. The government has certain rights in the invention.

### BACKGROUND

An important challenge in modern polymer chemistry is the efficient and controlled synthesis of polymers with unique topologies. Perhaps the simplest shape change is to convert a linear polymer into its cyclic analogue. By simply tying together the chain ends of a linear polymer it is possible to dramatically alter its physical properties. The repeat unit, functional groups, molecular weight (minus end groups), and polydispersity all remain the same when the chain ends are removed to create a cyclic polymer; however, the density, refractive index, T_{g}, viscoelasticity, reptation, and surface properties all change.

A common method for the synthesis of cyclic polymers involves intramolecular coupling of polymer chain ends; however, inherent limitations of this method are the requirement of dilute conditions and long reaction times. Ring closing methods continue to improve and provide an opportunity to obtain samples of unique ring-shaped polymers, though large scale production via ring closure is unlikely.

Thus, metal catalysts that polymerize alkenes via ring expansion metathesis polymerization (REMP) to yield cyclic polyalkenes are desirable.

### SUMMARY

Provided herein is a catalyst having a structure represented by formula (I), or dimer thereof:

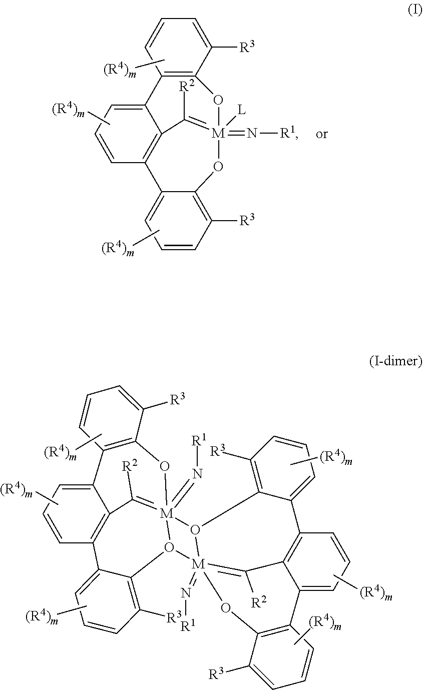

wherein
M is a transition metal;
L is absent or selected from the group of NH₃, N(R⁵)₃, Ar¹, C₁₋₆hydroxyalkyl, N≡C—R⁵, N═C(R⁵)₂, OR⁵, O(R⁵)₂, P(R⁵)₃, P(OR⁵)₃, PR⁵(OR⁵)₂, PR⁵₂(OR⁵), R⁵(CO)N(R⁵)₂, R⁵(CO)NHR⁵, R⁵CHO, R⁵COR⁵, R⁵COOR⁵, SR⁵, and S(R⁵)₂;
R¹ is selected from H, N═N—R^{a}, C₁-C₂₀alkyl, C₃₋₈cycloalkyl, C₁-C₂₀haloalkyl, Si—(R⁵)₃, and Ar¹, and R^{a} is selected from H, C₁-C₂₀alkyl, C₃₋₈cycloalkyl, and Ar¹;
R² is selected from H, Ar¹, C₁-C₂₀alkyl, C₅-C₈cycloalkyl, halo, C₁₋₂₀haloalkyl, NH₂, N(C₁-C₂₀alkyl)₂, NH(C₁-C₂₀alkyl), NHAr¹, N(Ar¹)(C₁₋₂₀alkyl), N(Ar¹)₂, OH, OAr¹, and O(C₁-C₂₀alkyl);
each R³ is independently selected from H, Ar¹, C₁-C₂₀alkyl, C₅-C₈cycloalkyl, halo, C₁-C₂₀haloalkyl, COOH, COOC₁-C₂₀alkyl, NH₂, N(C₁-C₂₀alkyl)₂, NH(C₁-C₂₀alkyl), NHAr¹, N(Ar¹)(C₁₋₂₀alkyl), N(Ar¹)₂, SH, OAr¹, O(C₁-C₂₀alkyl), and OH, or R³ together with an adjacent R⁴ and the carbon atoms to which they are attached form a five- to eight-membered cyclic group;
each m is independently 0, 1, 2, or 3;
each R⁴ is independently selected from Ar¹, C₁-C₂₀alkyl, C₅-C₈cycloalkyl, C₁-C₂₀haloalkyl, COOH, COOC₁-C₂₀alkyl, NH₂, N(C₁-C₂₀alkyl)₂, NH(C₁-C₂₀alkyl), NHAr¹, N(Ar¹)(C₁₋₂₀alkyl), N(Ar¹)₂, SH, halo, and OH, or two adjacent R⁴, together with the carbon atoms to which they are attached, can form a five- to eight-membered cyclic group;
each R⁵ is independently selected from H, C₁-C₂₀alkyl, C₅-C₈cycloalkyl, Ar¹, or two R⁵, together with the atoms to which they are attached, form a five- to eight-membered heterocycle comprising from 1 to 3 ring heteroatoms selected from O, N, and S; and
each Ar¹ is independently selected from C₀-C₃alkylene-C₆₋₂₀ aryl, or a 5-12 membered heteroaryl comprising from 1 to 3 ring heteroatoms selected from N, O, and S.

Also provided herein are methods of preparing the catalyst of formula (I), and dimers thereof, according to the disclosure, comprising:
reacting a compound of formula (II) and an organoazide having a structure of formula (III) under conditions sufficient to form the catalyst:

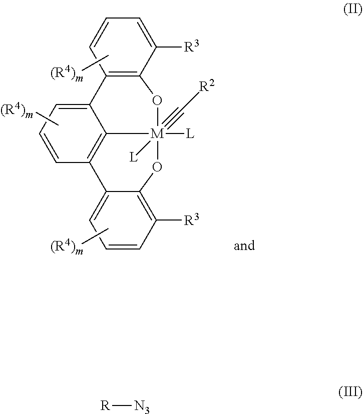

wherein in formula (II):
M is a transition metal;
each L is independently absent or selected from the group of NH₃, N(R⁵)₃, Ar¹, C₁₋₆hydroxyalkyl, N≡C—R⁵, N═C(R⁵)₂, OR⁵, O(R⁵)₂, P(R⁵)₃, P(OR⁵)₃, PR⁵(OR⁵)₂, PR⁵₂(OR⁵), R⁵(CO)N(R⁵)₂, R⁵(CO)NHR⁵, R⁵CHO, R⁵COR⁵, R⁵COOR⁵, SR⁵, and S(R⁵)₂;
R² is selected from H, Ar¹, C₁₋₂₀alkyl, C₅-C₈cycloalkyl, halo, C₁₋₂₀haloalkyl, NH₂, N(C₁₋₂₂ alkyl)₂, NH(C₁₋₂₂ alkyl), NHAr¹, N(Ar¹)(C₁₋₂₀alkyl), N(Ar¹)₂, OAr¹, O(C₁₋₂₂ alkyl), and Si—(R⁵)₃;
each R³ is independently selected from H, Ar¹, C₁-C₂₀alkyl, C₅-C₈cycloalkyl, halo, C₁-C₂₀haloalkyl, COOH, COOC₁₋₂₀alkyl, NH₂, N(C₁₋₂₀alkyl)₂, NH(C₁₋₂₀alkyl), NHAr¹, N(Ar¹)(C₁₋₂₀alkyl), N(Ar¹)₂, SH, OAr¹, O(C₁₋₂₀alkyl), and OH, or R³ together with an adjacent R⁴ and the carbon atoms to which they are attached form a five- to eight-membered cyclic group;
each R⁴ is independently selected from Ar¹, C₁-C₂₀alkyl, C₅-C₈cycloalkyl, C₁-C₂₀haloalkyl, COOH, COOC₁₋₂₀alkyl, NH₂, N(C₁₋₂₀alkyl)₂, NH(C₁₋₂₀alkyl), NHAr¹, N(Ar¹)(C₁₋₂₀alkyl), N(Ar¹)₂, SH, halo, and OH, or two adjacent R⁴, together with the carbon atoms to which they are attached, can form a five- to eight-membered cyclic group;
each R⁵ is independently selected from H, C₁-C₂₀alkyl, C₅-C₈cycloalkyl, Ar¹, or two R⁵, together with the atoms to which they are attached, form a five- to eight-membered heterocycle comprising from 1 to 3 ring heteroatoms selected from O, N, and S; and
each Ar¹ is independently selected from C₆₋₂₀aryl, C₁₋₃alkylene-C₆₋₂₀aryl, or a 5-12 membered heteroaryl comprising from 1 to 3 ring heteroatoms selected from O, N, and S; and
each m is independently 0, 1, 2, or 3; and
   wherein in formula (III):
   R is selected from H, C₁₋₂₀alkyl, C₃₋₈cycloalkyl, Si—(R⁵)₃, and Ar¹.

Also provided herein are methods of preparing a cyclic polymer, comprising:
admixing a plurality of alkene monomers, alkyne monomers, or both in the presence of the catalyst of formula (I), or dimers thereof, under conditions sufficient to polymerize the plurality of alkene monomers, alkyne monomers, or both to form the cyclic polymer, wherein the cyclic polymer ring comprises alkene groups.

Further aspects and advantages will be apparent to those of ordinary skill in the art from a review of the following detailed description. While the compositions and methods are susceptible of embodiments in various forms, the description hereafter includes specific embodiments with the understanding that the disclosure is illustrative, and is not intended to limit the disclosure to the specific embodiments described herein.

### BRIEF DESCRIPTION OF THE DRAWINGS

FIG. 1A depicts a graph of the % conversion of norbornene vs time for catalysts 1-Ph and 2-Ph, via ¹H NMR spectroscopy.

FIG. 1B depicts a zoomed-in view of FIG. 1A between 0 min to 60 min.

FIG. 2 depicts a graph of the Log of M_{W} versus elution volume for cyclic poly(norbornene) formed according to an embodiment of the invention and linear poly(norbornene).

FIG. 3 depicts a graph of the Log of [η] versus Log of M_{w} for cyclic poly(norbornene) formed according to an embodiment of the invention and linear poly(norbornene).

FIG. 4 depicts a graph of the mean square radius (<R_{g}²>) versus M_{w} for cyclic poly(norbornene) formed according to an embodiment of the invention and linear poly(norbornene).

FIG. 5 depicts Eyring plots for the conversion of catalyst 1-R to catalyst 2-R, between 70° C. to 100° C. for 1-Bn and 60° C. to 90° C. for 1-Ph, 1-Oct, and 1-Cy.

FIG. 6 depicts two predicted deazotation transition states for expulsion of N₂ from azoimido catalyst 1-Ph.

FIG. 7 depicts a graph of the calculated potential energy surface for the conversion of catalyst 1-Ph to catalyst 2-Ph. Gibbs free energy in kcal/mol at 353 K and 1 atm; TPSSh-D0(SMD)/cc-pVTZ-(PP)//TPSSh/cc-pVDZ-(PP) level of theory.

### DETAILED DESCRIPTION

Provided herein are compounds having a structure represented by formula (I) and methods of making and using said compounds. In embodiments, compounds having a structure represented by formula (I) can be in the form of a dimer. Compounds having a structure represented by formula (I), and dimers thereof, can be used as a catalyst in the preparation of cyclic polymers. Advantageously, compounds having a structure represented by formula (I), or dimers thereof, can generate high-molecular weight cyclic polyalkenes.

The compounds of the disclosure have structures represented by formulas (I), (II), and (III), and these compounds may also be referred to as “compounds of formula (I),” “compounds of formula (II),” and “compounds of formula (III)”, herein, respectively. The compounds of formula (I) may also be referred to as a “catalyst of formula (I)” throughout the disclosure.

It is to be understood that the terminology used herein is to describe particular aspects only and is not intended to be limiting. As used in the specification and the claims, the term “comprising” can include the aspect of “consisting of.” Unless defined otherwise, all technical and scientific terms used herein have the same meaning as commonly understood by one of ordinary skill in the art to which the disclosed compositions and methods belong. In this specification and in the claims which follow, reference will be made to several terms which shall be defined herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events described or in any other order that is logically possible.

The use of the terms “a,” “an,” “the,” and similar referents in the context of the disclosure herein (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure herein and is not a limitation on the scope of the disclosure herein unless otherwise indicated. No language in the specification should be construed as indicating any non-claimed element as essential to the practice of the disclosure herein.

### Definitions

As used herein, the term “alkyl” refers to straight chained and branched saturated hydrocarbon groups containing one to thirty carbon atoms, for example, one to twenty two carbon atoms, or one to twenty carbon atoms, or one to ten carbon atoms. The term Cn means the alkyl group has “n” carbon atoms. For example, C₄ alkyl refers to an alkyl group that has 4 carbon atoms. C₁₋₇alkyl and C₁-C₇ alkyl refer to an alkyl group having a number of carbon atoms encompassing the entire range (i.e., 1 to 7 carbon atoms), as well as all subgroups (e.g., 1-6, 2-7, 1-5, 3-6, 1, 2, 3, 4, 5, 6, and 7 carbon atoms). Nonlimiting examples of alkyl groups include, methyl, ethyl, n-propyl, isopropyl, n-butyl, sec-butyl (2-methylpropyl), t-butyl (1,1-dimethylethyl), 3,3-dimethylpentyl, and 2-ethylhexyl. Unless otherwise indicated, an alkyl group can be an unsubstituted alkyl group or a substituted alkyl group. The term “alkylene” refers to an alkyl group that is further substituted, e.g., alkylene-aryl refers to an alkyl group having an aryl substituent.

As used herein, the term “cycloalkyl” refers to an aliphatic cyclic hydrocarbon group containing three to eight carbon atoms (e.g., 3, 4, 5, 6, 7, or 8 carbon atoms). The term Cn means the cycloalkyl group has “n” carbon atoms. For example, C₅ cycloalkyl refers to a cycloalkyl group that has 5 carbon atoms in the ring. C₅₋₈ cycloalkyl and C₅-C₈ cycloalkyl refer to cycloalkyl groups having a number of carbon atoms encompassing the entire range (i.e., 5 to 8 carbon atoms), as well as all subgroups (e.g., 5-6, 6-8, 7-8, 5-7, 5, 6, 7, and 8 carbon atoms). Nonlimiting examples of cycloalkyl groups include cyclopropyl, cyclobutyl, cyclopentyl, cyclohexyl, cycloheptyl, and cyclooctyl. Unless otherwise indicated, a cycloalkyl group can be an unsubstituted cycloalkyl group or a substituted cycloalkyl group. The cycloalkyl groups described herein can be isolated or fused to another cycloalkyl group, a heterocycle group, an aryl group and/or a heteroaryl group.

As used herein, the term “heterocycle” is defined similarly as cycloalkyl, except the ring contains one to three heteroatoms independently selected from oxygen, nitrogen, and sulfur. In particular, the term “heterocycle” refers to a ring containing a total of three to eight atoms, of which 1, 2, 3 or three of those atoms are heteroatoms independently selected from the group consisting of oxygen, nitrogen, and sulfur, and the remaining atoms in the ring are carbon atoms. Nonlimiting examples of heterocycle groups include piperdine, tetrahydrofuran, tetrahydropyran, dihydrofuran, morpholine, and the like. Heterocycle groups can be saturated or partially unsaturated ring systems optionally substituted with, for example, one to three groups, independently selected alkyl, alkenyl, OH, C(O)NH₂, NH₂, oxo (═O), aryl, haloalkyl, halo, and OH. Heterocycle groups optionally can be further N-substituted with alkyl, hydroxyalkyl, alkylene-aryl, and alkylene-heteroaryl. The heterocycle groups described herein can be isolated or fused to another heterocycle group, a cycloalkyl group, an aryl group, and/or a heteroaryl group. When a heterocycle group is fused to another heterocycle group, then each of the heterocycle groups can contain three to eight total ring atoms, and one to three heteroatoms. In some embodiments, the heterocycle groups described herein comprise one oxygen ring atom (e.g., oxiranyl, oxetanyl, tetrahydrofuranyl, and tetrahydropyranyl).

As used herein, the term “aryl” refers to monocyclic or polycyclic (e.g., fused bicyclic and fused tricyclic) carbocyclic aromatic ring systems. Examples of aryl groups include, but are not limited to, phenyl, naphthyl, tetrahydronaphthyl, phenanthrenyl, biphenylenyl, indanyl, indenyl, anthracenyl, and fluorenyl. Unless otherwise indicated, an aryl group can be an unsubstituted aryl group or a substituted aryl group.

As used herein, the term “heteroaryl” refers to a cyclic aromatic ring having five to twelve total ring atoms (e.g., a monocyclic aromatic ring with 5-6 total ring atoms), and containing one to three heteroatoms selected from nitrogen, oxygen, and sulfur in the aromatic ring. Unless otherwise indicated, a heteroaryl group can be unsubstituted or substituted with one or more, and in particular one to four, substituents selected from, for example, halo, alkyl, alkenyl, OCF₃, NO₂, CN, NC, OH, alkoxy, amino, CO₂H, CO₂alkyl, aryl, and heteroaryl. In some cases, the heteroaryl group is substituted with one or more of alkyl and alkoxy groups. Heteroaryl groups can be isolated (e.g., pyridyl) or fused to another heteroaryl group (e.g., purinyl), a cycloalkyl group (e.g., tetrahydroquinolinyl), a heterocycle group (e.g., dihydronaphthyridinyl), and/or an aryl group (e.g., benzothiazolyl and quinolyl). Examples of heteroaryl groups include, but are not limited to, thienyl, furyl, pyridyl, pyrrolyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, and thiadiazolyl. When a heteroaryl group is fused to another heteroaryl group, then each ring can contain five or six total ring atoms and one to three heteroatoms in its aromatic ring.

As used herein, the term “cyclic group” refers to any ring structure comprising a cycloalkyl, heterocycle, aryl, heteroaryl, or a combination thereof. Unless otherwise indicated, a cyclic group can be an unsubstituted or a substituted cyclic group.

As used herein, the term “hydroxy” or “hydroxyl” refers to the “—OH” group. As used herein, the term “thiol” refers to the “—SH” group. As used herein, the term “hydroxyalkyl” refers to an alkyl group in which one or more of the hydrogen atoms are replaced by a hydroxyl group (OH). Such groups include but are not limited to, hydroxymethyl, hydroxyethyl, and the like.

As used herein, the term “alkoxy” or “alkoxyl” refers to a “—O-alkyl” group. As used herein, the term “aryloxy” or “aryloxyl” refers to a “—O-aryl” group.

As used herein, the term “ether” refers to an “alkyl-O-alkyl” group. The alkyl groups can together form a ring. A C₂-C₂₂ ether refers to an ether group wherein both alkyl groups together, or a ring formed therefrom have 2 to 22 carbons. When provided as a ligand, the ether can be coordinated to the metal center through the oxygen. As used herein, the term “thioether” is defined similarly to “ether” except the oxygen atom is replaced with a sulfur atom. As used herein, the term “polyether” refers to a “ether-(O-ether)ₙ” group, where n is greater than or equal to 1. As used herein, the term “polythioether” is defined similarly to “ether” except the oxygen atom is replaced with a sulfur atom.

As used herein, the term “halo” is defined as fluoro, chloro, bromo, and iodo. The term “haloalkyl” refers to an alkyl group that is substituted with at least one halogen, and includes perhalogenated alkyl (i.e., all hydrogen atoms substituted with halogen).

As used herein, the term “carboxy” or “carboxyl” refers to a “—COOH” group.

As used herein, the term “amino” refers to a —NH₂ group, wherein one or both hydrogen can be replaced with an alkyl, cycloalkyl, or aryl group. As used herein, the term “amido” refers to an amino group that is substituted with a carbonyl moiety (e.g., —NRC(═O)— or —OC(═O)—NR—), wherein R is a substituent on the nitrogen (e.g., alkyl or H). When referring to a ligand, the term “amine” refers to a —NH₃ group, where one, two, or three hydrogen can be replaced with an alkyl, cycloalkyl, or aryl group. When referring to a ligand, the term “amide” refers to a NR₂ group, wherein each R is independently a hydrogen, alkyl, cycloalkyl, or aryl group. As used herein “imine” refers to a —N(R)═CR₂ group, wherein each R is independently an alkyl, cycloalkyl, or aryl group.

As used herein, the term “phosphine” refers to a —PH₃ group, wherein one, two or three hydrogen can be replaced with an alkyl, cycloalkyl, or aryl group. As used herein “phosphite” refers to a —P(OR)₃ group, wherein each R can individually be alkyl, cycloalkyl, or aryl. As used herein, “phosphonite” refers to a —PR(OR)₂ group, wherein each R can individually be alkyl, cycloalkyl, or aryl. As used herein, “phosphinite” refers to a —PR₂(OR) group, wherein each R can individually be alkyl, cycloalkyl, or aryl.

As used herein, the term “ester” refers to a —C(═O)OR group, wherein R is a substituent on the oxygen (e.g., alkyl or aryl).

As used herein, the term “substituted,” when used to modify a chemical functional group, refers to the replacement of at least one hydrogen radical on the functional group with a substituent. Substituents can include, but are not limited to, alkyl, cycloalkyl, alkenyl, cycloalkenyl, alkynyl, heterocycle, ether, polyether, thioether, polythioether, aryl, heteroaryl, hydroxyl, oxo (═O), alkoxy, heteroalkoxy, aryloxy, heteroaryloxy, ester, thioester, carboxy, cyano, nitro, amino, amido, amide, and halo (e.g., fluoro, chloro, bromo, or iodo). When a chemical functional group includes more than one substituent, the substituents can be bound to the same carbon atom or to two or more different carbon atoms.

### Catalysts of the Disclosure

Provided herein are compounds having a structure represented by formula (I), or dimers thereof:

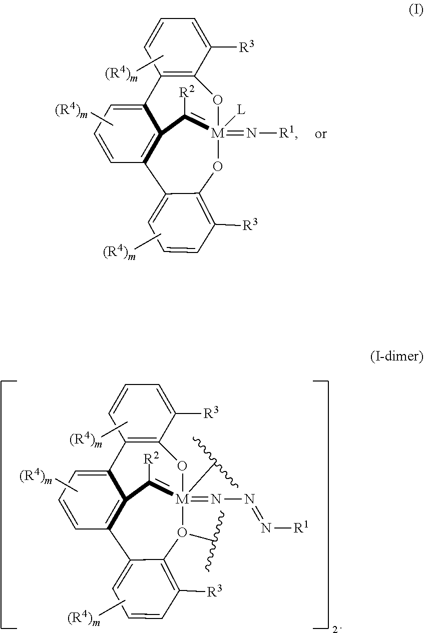

The compound having a structure represented by formula (I-dimer) can also be represented as:

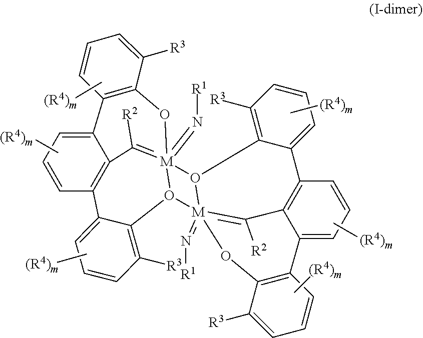

In general, in the compounds of the disclosure, each M is a transition metal. In embodiments, M is a group 6 transition metal. In embodiments, M is molybdenum (Mo) or tungsten (W). In embodiments, M is tungsten (W).

Throughout the disclosure, each metal-nitrogen bond is generally represented as a double bond. However, it will be understood that the metal-nitrogen bond can also have some triple bond character due to additional overlapping pi bonds. Thus, as used herein, in the structure of Formula (I) or dimers thereof, metal-nitrogen triple bonds (M≡N) are considered equivalent to metal-nitrogen double bonds (M═N).

In general, R² is selected from the group of H, Ar¹, C₁-C₂₀alkyl, C₅-C₈cycloalkyl, halo, C₁-C₂₀haloalkyl, NH₂, N(C₁-C₂₀alkyl)₂, NH(C₁-C₂₀alkyl), NHAr¹, N(Ar¹)(C₁₋₂₀alkyl), N(Ar¹)₂, OAr¹, O(C₁-C₂₀alkyl), and OH. In embodiments, R² is H, Ar¹, C₁-C₂₀alkyl, C₅-C₈cycloalkyl, or C₁-C₂₀haloalkyl. In embodiments, R² is H, C₁-C₂₀haloalkyl or C₁-C₂₀alkyl. In embodiments, R² is tert-butyl.

In general, each occurrence of R³ is independently selected from the group of H, Ar¹, C₁-C₂₀alkyl, C₅-C₈cycloalkyl, halo, C₁-C₂₀haloalkyl, COOH, COOC₁-C₂₀alkyl, NH₂, N(C₁-C₂₀alkyl)₂, NH(C₁-C₂₀alkyl), NHAr¹, N(Ar¹)(C₁₋₂₀alkyl), N(Ar¹)₂, SH, OAr¹, O(C₁-C₂₀alkyl), and OH, or R³ together with an adjacent R⁴ and the carbon atoms to which they are attached form a five- to eight-membered cyclic group. In embodiments, each R³ is independently selected from H, Ar¹, C₁-C₂₀alkyl, C₅-C₈cycloalkyl, halo, C₁-C₂₀haloalkyl, COOH, COOC₁-C₂₀alkyl, NH₃, SH, and OH. In embodiments, each R³ is independently selected from H, Ar¹, C₁-C₂₀haloalkyl or C₁-C₂₀alkyl. In embodiments, at least one R³ is tert-butyl. In embodiments, each R³ is tert-butyl.

In general, each occurrence of R⁴ is independently selected from the group of Ar¹, C₁-C₂₀alkyl, C₅-C₈cycloalkyl, C₁-C₂₀haloalkyl, COOH, COOC₁-C₂₀alkyl, NH₂, N(C₁-C₂₀alkyl)₂, NH(C₁-C₂₀alkyl), NHAr¹, N(Ar¹)(C₁₋₂₀alkyl), N(Ar¹)₂, SH, halo, and OH, or two adjacent R⁴, together with the carbon atoms to which they are attached, can form a five- to eight-membered cyclic group. In embodiments, each R⁴ is independently selected from C₁-C₂₀alkyl, C₅-C₈cycloalkyl, Ar¹, COOH, COOC₁-C₂₀alkyl, NH₃, SH, halo, C₁-C₂₀haloalkyl, and OH. In embodiments, each R⁴ C₁-C₂₀alkyl, Ar¹, C₅-C₈cycloalkyl, or C₁-C₂₀haloalkyl.

In general, m can be 0, 1, 2, or 3. In embodiments, each m is 0. In embodiments, at least one m is 1. In embodiments, at least one m is 2. In embodiments, at least two m are 1.

In general, each L is independently absent or can be an L-type ligand. In embodiments, each L is independent absent or can be selected from the group of phosphine, phosphite, phosphonite, phosphinite, amine, amide, imine, alkoxy, ether, thioether, and a five- or six-membered monocyclic group having 1 to 3 ring heteroatoms; or both L together comprise a bidentate ligand. L-type ligands are described in detail throughout Gray L. Spessard and Gray O. Miessler, Organometallic Chemistry, published by Oxford University Press, 2010, for example, on page 59, which is herein incorporated by reference. Suitable bidentate ligands include, but are not limited to, bipyridine, ethylenediamine, diaminocyclohexane, acetylacetonate, oxalate, and phenanthroline. The five- or six-membered monocyclic groups can include 1 to 3 heteroatoms or 1 to 2 heteroatoms, for example, pyridine, pyridazine, pyrimidine, pyrazine, triazine, pyrrole, pyrazole, imidazoletriazole, pyran, pyrone, dioxin, and furan. The five- or six-membered monocyclic groups can be substituted with halo, C₁-C₂₂alkyl, substituted C₁-C₂₂alkyl, C₁-C₂₂heteroalkyl, substituted C₁-C₂₂heteroalkyl, C₆-C₂₄aryl, substituted C₆-C₂₄ aryl, C₆-C₂₄heteroaryl, substituted C₆-C₂₄ heteroaryl, and functional groups, including but not limited to, C₁-C₂₂ alkoxy, C₆-C₂₄ aryloxy, C₂-C₂₂ alkylcarbonyl, C₆-C₂₄ arylcarbonyl, carboxy, carboxylate, carbamoyl, carbamido, formyl, thioformyl, amino, nitro, and nitroso.

In embodiments, L is absent or selected from the group of NH₃, N(R⁵)₃, Ar¹, C₁₋₆hydroxyalkyl, N≡C—R⁵, N═C(R⁵)₂, OR⁵, O(R⁵)₂, P(R⁵)₃, P(OR⁵)₃, PR⁵(OR⁵)₂, PR⁵₂(OR⁵), R⁵(CO)N(R⁵)₂, R⁵(CO)NHR⁵, R⁵CHO, R⁵COR⁵, R⁵COOR⁵, SR⁵, and S(R⁵)₂. In embodiments, L is Ar¹, N(R⁵)₃, P(R⁵)₃, S(R⁵)₂ or O(R⁵)₂. In embodiments, L is tetrahydrofuran (THF), pyridine, or thiophene.

In general, R¹ is independently selected from the group of H, N═N—R^{a}, C₁-C₂₀alkyl, C₃₋₈cycloalkyl, C₁-C₂₀haloalkyl, Si—(CH₃)₃, and Ar¹. In embodiments, R¹ is H, phenyl, Si—(CH₃)₃, n-octyl, cyclohexyl, or benzyl. In embodiments, R¹ is N═N—R^{a}.

In general, R^{a} is independently selected from the group of H, C₁-C₂₀alkyl, C₃₋₈cycloalkyl, C₁₋₂₀haloalkyl, and Ar¹. In embodiments, R^{a} is C₁-C₂₀alkyl, C₃₋₈cycloalkyl, or Ar¹. In embodiments, R^{a} is phenyl, benzyl, n-octyl, or cyclohexyl.

In general, each occurrence of R⁵ is independently selected from the group of H, C₁-C₂₀alkyl, C₅-C₈cycloalkyl, and Ar¹, or two R⁵, together with the atoms to which they are attached, form a five- to eight-membered heterocycle comprising from 1 to 3 ring heteroatoms selected from O, N, and S. In embodiments, each R⁵ of L is independently selected from C₁-C₂₀alkyl, C₅-C₈cycloalkyl, and Ar¹.

In general, each occurrence of Ar¹ is independently selected from C₆-C₂₀aryl, C₁-C₃alkylene-C₆-C₂₀aryl, or a 5-12 membered heteroaryl comprising from 1 to 3 ring heteroatoms selected from O, N, and S. In embodiments, each Ar¹ is C₆-C₂₀aryl, or C₁-C₃alkylene-C₆-C₂₀aryl. In embodiments, each Ar¹ of R³ is C₆₋₂₀aryl or C₁₋₃alkylene-C₆₋₂₀aryl. In embodiments, each Ar¹ of R⁴ is C₆₋₂₀aryl or C₁₋₃alkylene-C₆₋₂₀aryl. In embodiments, each Ar¹ of R⁵ is C₆₋₂₀aryl or C₁₋₃alkylene-C₆₋₂₀aryl. In embodiments, each Ar¹ of R¹ is C₀₋₃alkylene-C₆₋₂₀aryl.

### Methods of Making the Catalysts of the Disclosure

Further provided herein are methods for making the compound having a structure represented by formula (I) or dimers thereof, comprising reacting a compound of formula (II) and an organoazide having a structure of formula (III):

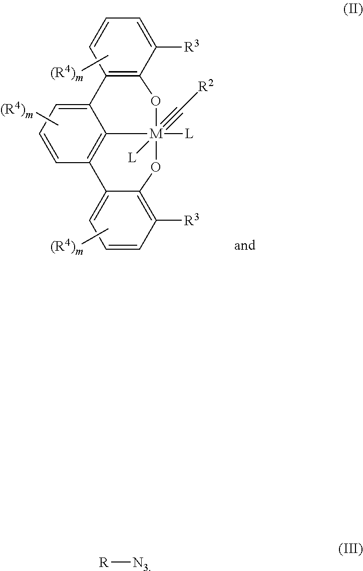

In general, L is as described above. In embodiments, at least one L is absent. In embodiments wherein L is absent, the compound of formula (II) is coordinatively unsaturated. In embodiments, at least one L is a phosphine. In embodiments, at least one L is an amine. In refinements of the foregoing embodiments, at least one L is selected from NH₂, dimethyl amine and diethyl amine. In embodiments, at least one L is an ether. In embodiments at least one L is a five- or six-membered monocyclic group having 1 to 3 ring heteroatoms. In refinements of the foregoing embodiment, at least one L is selected from tetrahydrofuran, tetrahydrothiophene, pyridine, and tetrahydropyran. In some embodiments, both L together comprise a bidentate ligand selected from bipyridine, ethylenediammine, diaminocyclohexane, acetylacetonate, oxalate, and phenanthroline.

In embodiments, L can comprise THF and derivatives thereof, thiophene and derivatives thereof, or pyridine and derivatives thereof. In embodiments, both L's are present and each L is THF.

In general, each occurrence of R can comprise H, C₁₋₂₀alkyl, C₃₋₈cycloalkyl, Si—(R⁵)₃, or Ar¹.

In general, each occurrence of R² can comprise H, Ar¹, C₁₋₂₀alkyl, C₅-C₈cycloalkyl, halo, C₁₋₂₀haloalkyl, NH₂, N(C₁₋₂₂ alkyl)₂, NH(C₁₋₂₂ alkyl), NHAr¹, N(Ar¹)(C₁₋₂₀alkyl), N(Ar¹)₂, OAr¹, O(C₁₋₂₂ alkyl), or OH. In embodiments, R² is H, Ar¹, C₁-C₂₀alkyl, C₅-C₈cycloalkyl, or C₁-C₂₀haloalkyl. In embodiments, R² is H, C₁-C₂₀haloalkyl or C₁-C₂₀alkyl. In embodiments, R² is tert-butyl.

In general, each occurrence of R³ can comprise H, Ar¹, C₁-C₂₀alkyl, C₅-C₈cycloalkyl, halo, C₁-C₂₀haloalkyl, COOH, COOC₁₋₂₀alkyl, NH₂, N(C₁₋₂₀alkyl)₂, NH(C₁₋₂₀alkyl), NHAr¹, N(Ar¹)(C₁₋₂₀alkyl), N(Ar¹)₂, SH, OAr¹, O(C₁₋₂₀alkyl), OH, or R³ together with an adjacent R⁴ and the carbon atoms to which they are attached form a five- to eight-membered cyclic group. In embodiments, each R³ is independently selected from H, Ar¹, C₁-C₂₀alkyl, C₅-C₈cycloalkyl, halo, C₁-C₂₀haloalkyl, COOH, COOC₁-C₂₀alkyl, NH₃, SH, and OH. In embodiments, each R³ is independently selected from H, Ar¹, C₁-C₂₀haloalkyl or C₁-C₂₀alkyl. In embodiments, at least one R³ is tert-butyl. In embodiments, R³ is tert-butyl.

In general, each occurrence of R⁴ can comprise Ar¹, C₁-C₂₀alkyl, C₅-C₈cycloalkyl, C₁-C₂₀haloalkyl, COOH, COOC₁₋₂₀alkyl, NH₂, N(C₁₋₂₀alkyl)₂, NH(C₁₋₂₀alkyl), NHAr¹, N(Ar¹)(C₁₋₂₀alkyl), N(Ar¹)₂, SH, halo, OH, or two adjacent R⁴, together with the carbon atoms to which they are attached, can form a five- to eight-membered cyclic group. In embodiments, each R⁴ is independently selected from C₁-C₂₀alkyl, C₅-C₈cycloalkyl, Ar¹, COOH, COOC₁-C₂₀alkyl, NH₃, SH, halo, C₁-C₂₀haloalkyl, and OH. In embodiments, each R⁴ is C₁-C₂₀alkyl, Ar¹, C₅-C₈ cycloalkyl, or C₁-C₂₀haloalkyl.

In general, each occurrence of Ar¹ can comprise C₆₋₂₀aryl, C₁₋₃alkylene-C₆₋₂₀aryl, or a 5-12 membered heteroaryl comprising from 1 to 3 ring heteroatoms selected from O, N, and S.

In general, m can be 0, 1, 2, or 3. In embodiments, each m is 0. In embodiments, at least one m is 1. In embodiments, at least one m is 2. In embodiments, at least two m are 1.

In general, each occurrence of R⁵ is independently selected from the group of H, C₁-C₂₀alkyl, C₅-C₈cycloalkyl, and Ar¹, or two R⁵, together with the atoms to which they are attached, form a five- to eight-membered heterocycle comprising from 1 to 3 ring heteroatoms selected from O, N, and S. In embodiments, each R⁵ of L is independently selected from C₁-C₂₀alkyl, C₅-C₈cycloalkyl, and Ar¹.

In embodiments, the reaction of the compound of formula (II) and the compound of formula (III) can occur neat, for example, in cases when the compound of formula (II) is a liquid. In embodiments, the reaction of the compound of formula (II) and the compound of formula (III) can occur in solution. Suitable solvents include nonpolar aprotic solvents, such as, but not limited to, benzene, toluene, hexanes, pentanes, dichloromethane, trichloromethane, chloro-substituted benzenes, deuterated analogs of the foregoing and combinations of any of the foregoing. As will be understood by one of ordinary skill in the art, polar aprotic solvents may also be suitable provided they do not compete with the organoazide of formula (III) to coordinate at the metal center. Suitable polar aprotic solvents can include, but are not limited to, diethyl ether, ethyl acetate, acetone, dimethylformamide, dimethoxyethane, tetrahydrofuran, acetonitrile, dimethyl sulfoxide, nitromethane, propylene carbonate, deuterated analogs of the foregoing, and combinations of the foregoing.

The reaction of the compound of formula (II) and the compound of formula (III) can occur at any suitable temperature for any suitable time. It is well understood in the art that the rate of a reaction can be controlled by tuning the temperature. Thus, in general, as the reaction temperature increases the reaction time can decrease.

Reaction temperatures can be in a range of about −80° C. to about 100° C., about −70 to about 80° C., about −50° C. to about 75° C., about −25° C. to about 50° C., about 0° C. to about 35° C., about 5° C. to about 30° C., about 10° C. to about 25° C., about 15° C. to about 25° C., or about 20° C. to about 25° C., for example, about 0° C., about 5° C., about 10° C., about 15° C., about 20° C., about 25° C., about 30° C., or about 35° C. Reaction times can be instantaneous or in a range of about 30 seconds to about 72 h, about 1 min to about 72 h, about 5 min to about 72 h, about 10 min to about 48 h, about 15 min to about 24 h, about 20 min to about 12 h, about 25 min to about 6 h, or about 30 min to about 3 h, for example, 30 seconds, 1 min, 5 min, 10 min, 15 min, 20 min, 25 min, 30 min, 35 min, 40 min, 45 min, 50 min, 55 min, 60 min, 75 min, 90 min, 105 min, 2 h, 3 h, 4 h, 5 h, 6 h, 12 h, 18 h, 24 h, 36 h, 48 h, 60 h, or 72 h.

In embodiments, reaction of the compound of formula (II) and the compound of formula (III) can be done under conditions sufficient to drive off nitrogen and prepare a compound of formula (I), wherein R¹ is selected from the group of H, C₁-C₂₀alkyl, C₃₋₈cycloalkyl, C₁-C₂₀haloalkyl, and Ar¹.

In alternative embodiments, the reaction of the compound of formula (II) and the compound of formula (III) can be done under conditions sufficient to prepare a compound of formula (I), wherein R¹ is N═N—R^{a}. Such a compound of formula (I) can have a structure according to formula (I′):

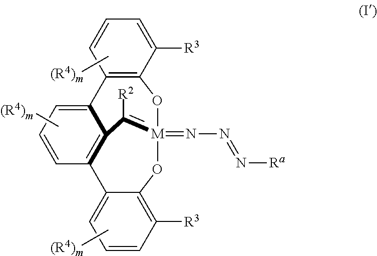

wherein each of R^{a}, R², R³, R⁴, and m are defined as defined herein for compounds of formula (I). It will be understood that compounds of formula (I′) are a subset of compounds of formula (I), wherein R¹ is N═N—R^{a}. In embodiments, the methods of the disclosure further provide converting a compound of formula (I′) to a compound of formula (I), wherein R¹ selected from the group of H, C₁-C₂₀alkyl, C₃₋₈cycloalkyl, C₁-C₂₀haloalkyl, Si—(R⁵)₃, and Ar¹.

The conditions sufficient to prepare a compound of formula (I) directly from a compound of formula (I) wherein R¹ is selected from the group of H, C₁-C₂₀alkyl, C₃₋₈cycloalkyl, C₁-C₂₀haloalkyl, Si—(R⁵)₃, and Ar¹ from the compound of formula (II) and organoazide having a structure of formula (III) are the same conditions sufficient to convert a compound of formula (I′) to a compound of formula (I) wherein R¹ is selected from the group of H, C₁-C₂₀alkyl, C₃₋₈cycloalkyl, C₁-C₂₀haloalkyl, Si—(R⁵)₃, and Ar¹, for example, by heating the mixture of compound (III) and compound (II) or heating compound (I′). Heating can take place in a solvent at a temperature of about 50° C., about 60° C., about 70° C., about 80° C., about 90° C., or about 100° C. Suitable solvents include any solvent disclosed herein as suitable for the reaction of the compound of formula (II) with the organoazide of formula (III).

### Methods of Preparing Polymers

The disclosure further provides a method of preparing a cyclic polymer, the method comprising admixing a plurality of alkene monomers, alkyne monomers, or both, in the presence of the catalyst of formula (I) or dimer thereof, herein under conditions sufficient to polymerize the plurality of alkene monomers, alkyne monomers, or both thereby forming the cyclic polymer, wherein the cyclic polymer ring comprises alkene groups.

Cyclic polymers can be prepared from any alkene monomer, i.e. any compound that includes a carbon-carbon double bond. In embodiments, more than one alkene monomer can be defined as the plurality of alkene monomers. A wide variety of alkene monomers, including, but not limited to, unsubstituted, monosubstituted, or disubstituted alkenes can be used to prepare cyclic polymers. Substituted alkenes can include alkenes substituted with 1 to 3 heteroatoms selected from oxygen, nitrogen, and sulfur. The alkene monomer can be a cyclic alkene. In embodiments, the cyclic alkene monomer can be bicyclic. In embodiments, the plurality of alkenes can comprise a mixture of different alkene monomers. In embodiments, the plurality of alkene monomers can comprise the same alkene monomers. The plurality of alkenes polymerized to form a cyclic polymer can comprise unsubstituted or substituted cyclopropene, cyclobutene, cyclopentene, cycloheptene, and cyclooctene, norbornene, dicyclopentadiene, norbornene anhydride, diester from norbornene anhydride, imide from norbornene anhydride, oxanorbornene, oxanorbornene anhydride, ester of oxanorbornene anhydride, and imide of oxanorbornene anhydride, or combinations thereof, wherein the ester is from a C₁-C₁₀ alkyl or aryl alcohol, the imide is from C₁-C₁₀ alkyl or aryl amine; wherein substituents can be C₁-C₁₀ alkyl, aryl, C₁-C₁₀ alkoxy, aryloxy, C₁-C₁₀ carboxylic acid ester, or carboxylic acid amide, optionally substituted one or two times with C₁-C₁₀ alkyl or aryl.

Cyclic polymers can be prepared from any alkyne monomer, i.e. any compound that includes a carbon-carbon triple bond. In embodiments, more than one alkyne monomer can be defined as the plurality of alkyne monomers. In embodiments, the alkyne monomer is selected from cyclooctyne, cycloocta-1,5-diyne, phenylacetylene, and

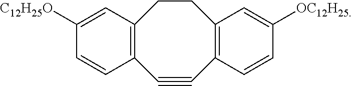

Examples of solvents that may be used in the polymerization reaction include organic, protic, or aqueous solvents that are inert under the polymerization conditions, such as aromatic hydrocarbons, halogenated hydrocarbons, ethers, aliphatic hydrocarbons, alcohols, water, or mixtures thereof. Suitable halogenated hydrocarbon solvents include methylene chloride, chloroform, chlorobenzene, 1,2-dichloroethane, dichlorobenzene, and mixtures thereof.

The polymerization can be carried out at, for example, ambient temperatures at dry conditions under an inert atmosphere. The polymerization can be carried out at a temperature in the range of about 0° C. to about 100° C. or greater, for example, in a range of about 10° C. to about 60° C. or about 20° C. to about 40° C. Polymerization times will vary, depending on the particular monomer, metallacyclopropene compound, and desired molecular weight of the cyclic polymer product. The progress of the reaction can be monitored by standard techniques, e.g., nuclear magnetic resonance (NMR) spectroscopy.

The molecular weight of the cyclic polymers can be small, equivalent to oligomers of three to ten repeating units, or the molecular weights can be of any size up to tens and hundreds of thousands or millions in molecular weight, for example, in a range of about 200 Da to about 5,000,000 Da, about 500 Da to about 4,000,000 Da, about 1,000 Da to about 3,000,000 Da, about 5,000 Da to about 2,000,000 Da or about 10,000 to about 1,000,000 Da. The cyclic polyalkene can be converted to substituted cyclic polyalkanes by an addition reaction at the alkene groups of the cyclic polyalkenes; for example, the addition of halogens, alcohols, amines, or any other olefin addition reactions.

The cyclic polymers can display one or more geometries across the polymer backbone. Tacticity can be determined with ¹H and ¹³C{¹H} NMR spectroscopy at low conversion and post-polymerization modification of the cyclic polymer. Generally, the cyclic polymers are syndiotactic. In embodiments, the cyclic polymer is syndiotactic. In embodiments, the alkene groups of the cyclic polymer are at least 50%, at least 60%, at least 70%, at least 80%, or at least 90% cis. In embodiments, the cyclic polymer is syndiotactic and at least 90% of the alkene groups are cis.

Following polymer synthesis and recovery, the unsaturated polymer provided may be hydrogenated using conventional means, e.g., via standard H₂/Pd/C procedures or via tosyl-hydrazine decomposition. Generally, either procedure will result in a saturated polymer having hydrogenated more than 99% of the unsaturated functionalities in the polymer backbone, as may be determined by ¹H and ¹³C{¹H} NMR spectroscopy. In embodiments, the method herein can further comprise hydrogenating the cyclic polymer alkene groups to form cyclic polymer alkane groups. In embodiments, the hydrogenation is performed under conditions sufficient to fully hydrogenate the cyclic polymer alkene groups, thereby forming a saturated cyclic polymer. As used herein, a cyclic polymer is fully hydrogenated if more than 99% of the unsaturated functionalities are hydrogenated. Advantageously, the stereoregularity of the polymers are maintained during hydrogenation, providing for stereoregular saturated cyclic polymers.

### EXAMPLES

### Materials and Methods

Unless specified otherwise, all manipulations were performed under an inert atmosphere using glove-box techniques. Toluene and pentane were dried using a GlassCountour drying column and stored over 3 Å molecular sieves. Benzene-d₆ (Cambridge Isotopes) was dried over calcium hydride (CaH₂), distilled, and stored over 3 Å molecular sieves. [^{t}BuOCO]W≡C^{t}Bu(THF)₂ (Complex A) was prepared according to literature procedure (Sarkar, S. et al., J. Am. Chem. Soc. 2012, 134, 4509-4512). Linear cis-syndiotactic polynorbornene (cis-poly(NBE)) was synthesized following a literature procedure (Grubbs et al., J. Am. Chem. Soc. 2016, 138, 1394-1405), using the commercially available catalyst, complex 15 having a structure of

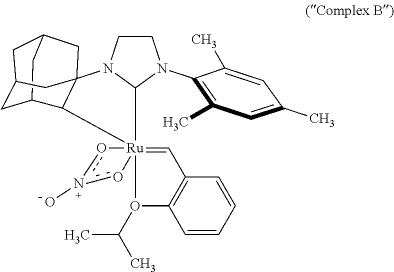

(Grubbs et al., J. Am. Chem. Soc. 2013, 135, 10032-10035). Complex B was purchased from Sigma-Aldrich (CAS 1352916-84-7, Hoveyda-Grubbs Catalyst M2001) and used without further purification. Post polymerization bromination of poly(NBE) was conducted according to literature procedure (Hyvl et al., Macromolecules 2015, 48, 3148-3152). NMR spectra were obtained on Varian INOVA 500 MHz and Varian INOVA2 500 MHz spectrometers, or equivalent. Chemical shifts are reported in δ (ppm). For ¹H and ¹³C{¹H}NMR spectra, the residual solvent peaks were used as an internal reference. Molecular weight, radius of gyration and polydispersity were determined by size exclusion chromatography (SEC) in dimethylacetamide (DMAc) with 50 mM LiCl at 50° C. and a flow rate of 1.0 mL/min (Agilent isocratic pump, degasser, and auto-sampler, columns: PLgel 5 μm guard+two ViscoGel I-series G3078 mixed bed columns: molecular weight range 0-20×10³+ and 0-100×10⁴ g mol⁻¹). Detection consisted of a Wyatt Optilab T-rEX refractive index detector, or equivalent, operating at 658 nm and a Wyatt miniDAWN Treos light scattering detector, or equivalent, operating at 659 nm. Absolute molecular weights and polydispersities were calculated using Wyatt ASTRA software or equivalent.

### Example 1: Synthesis of Catalyst 1

### General Procedure

In a vial equipped with a magnetic stir bar, Complex A, [^{t}BuOCO]W=C^{t}Bu(THF)₂ (0.13 mmol) was dissolved in 5 mL of benzene. An aliquot of the appropriate azide, N₃—R (0.14 mmol) was added at room temperature. In these preparations, R was one of phenyl, benzyl, n-octyl, or cyclohexyl. After the appropriate reaction time, the solution was dried under vacuum yielding catalyst 1-R.

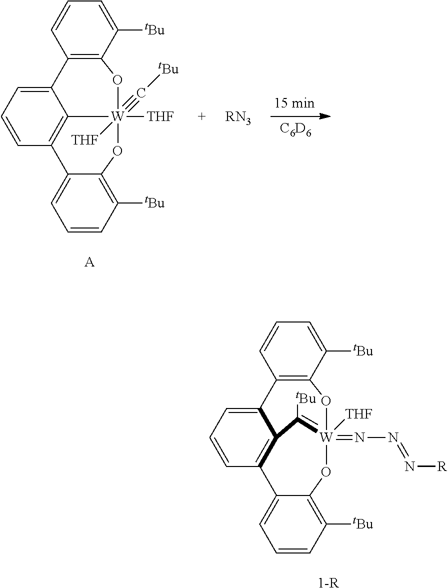

| | R group | Reaction Time | Solvent; (Volume) |
|---|---|---|---|
| 1-Ph | Phenyl | 15 minutes | Benzene; (5 mL) |
| 1-Bn | Benzyl | 15 minutes | Benzene; (5 mL) |
| 1-Oct | n-Octyl | 15 minutes | Benzene; (5 mL) |
| 1-Cy | Cyclohexyl | 15 minutes | Benzene; (5 mL) |

### Synthesis and Characterization of 1-Ph

In a vial equipped with a stir bar, complex A (100 mg, 0.13 mmol) was dissolved in 5 mL of benzene, phenyl azide (16.7 mg, 15.4 μL, 0.14 mmol) was added in the above solution at room temperature. An immediate color change from dark red to yellowish orange was observed. The reaction was then stirred at room temperature for 15 minutes. Volatiles were removed under vacuum yielding catalyst 1-Ph.

¹H NMR (C₆D₆, 400 MHz) δ (ppm): 7.90 (m, 2H, Ar—H_{36,40}), 7.42 (d, 2H, J=8 Hz, Ar—H_{8,10}), 7.36 (dd, 2H, J₁=4 Hz, J₂=8 Hz, Ar—H_{3,16}), 7.32 (dd, 2H, J₁=4 Hz, J₂=8 Hz, Ar—H_{5,14}), 7.21 (t, 1H, J=8 Hz, Ar—H₉), 7.08 (m, 2H, Ar—H_{37,39}), 7.03 (m, 1H, Ar—H₃₈), 6.86 (dd, 2H, J₁=J₂=8 Hz, Ar—H_{4,15}), 3.58 (m, 4H, THF—H_{32,35}), 1.58 (s, 18H, Ar-^{t}Bu, H_{20-22,24-26}), 1.05 (m, 4H, THF—H_{33,34}), 0.96 (s, 9H, W═CC(CH₃)₃, H₂₉₋₃₁). The number labels in the structure below correspond to the number labels for each resonance reported.

¹³C{¹H} NMR (C₆D₆, 400 MHz) δ (ppm): 274.94 (s, W═C_{α}, C₂₇), 169.36 (s, C_{1,18}), 152.14 (s, C_{7,11}), 150.61 (s, C₄₁), 138.84 (s, C_{2,17}), 133.37 (s, C₉), 131.38 (s, C_{6,13}), 129.58 (s, C₃₈), 129.54 (s, C_{37,39}), 128.68 (s, C_{5,14}), 126.67 (s, C_{3,16}), 123.00 (s, C₁₂), 122.26 (s, C_{36,40}), 119.72 (s, C_{4,15}), 71.98 (s, C_{32,35}), 47.41 (s, C₂₈), 35.51 (s, C_{19,23}), 34.40 (s, C₂₉₋₃₁), 30.68 (s, C_{20-22, 24-26}), 25.24 (s, C_{33,34}). The number labels in the structure below correspond to the number labels for each resonance reported.

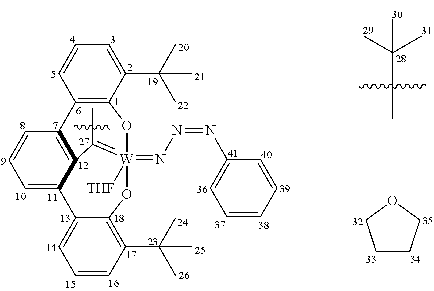

### Characterization of 1-Bn

In a vial equipped with a stir bar, complex A (100.0 mg, 0.13 mmol) was dissolved in 5 mL of benzene, then benzyl azide (18.6 mg, 17.5 μL, 0.14 mmol) was added at room temperature. An immediate color change from dark red to light yellow was observed. The resultant solution was then stirred at room temperature for 15 min. All volatiles were removed under vacuum yielding 1-Bn.

¹H NMR (C₆D₆, 400 MHz) δ (ppm): 7.42 (d, 2H, J=7.7 Hz, Ar—H_{8,10}), 7.39 (dd, 2H, J₁=1.8 Hz, J₂=7.9 Hz, Ar—H_{3,16}), 7.33 (dd, 2H, J₁=1.8 Hz, J₂=7.9 Hz, Ar—H_{5,14}), 7.29 (m, 2H, Ar—H_{38,42}), 7.22 (t, 1H, J=7.7 Hz, Ar—H₉), 7.16 (m, 2H, Ar—H_{39,41}), 7.06 (m, 1H, Ar—H₄₀), 6.87 (t, 2H, J=7.9 Hz, Ar—H_{4,16}), 4.82 (s, 2H, Bz-H₃₆), 3.56 (m, 4H, THF—H_{32,35}), 1.59 (s, 18H, Ar-^{t}Bu, H_{20-22,24-26}), 1.23 (m, 4H, THF—H_{33,34}), 0.90 (s, 9H, W═CC(CH₃)₃, H₂₉₋₃₁). The number labels in the structure below correspond to the number labels for each resonance reported.

¹³C{¹H}NMR (C₆D₆, 400 MHz) δ (ppm): 274.25 (s, W═Cα), 169.25 (s, C_{1,18}), 151.56 (s, C_{7,11}), 138.79 (s, C_{2,17}), 136.71 (s, C₃₇), 133.01 (s, C₉), 131.54 (s, C_{6,13}), 129.49 (s, C_{8,10}), 129.36 (s, C_{5,14}), 128.71 (s, C_{39,41}), 128.65 (s, C₄₀), 127.76 (s, C_{38,42}), 127.52 (s, C₁₂), 126.63 (s, C_{3,16}), 119.66 (s, C_{4,15}), 71.90 (s, C_{32,35}), 66.86 (s, C₃₆), 47.38 (s, C₂₈), 35.52 (s, C_{19,23}), 34.35 (s, C₂₉₋₃₁), 30.76 (s, C_{20-22,24-26}), 25.24 (s, C_{33,34}). The number labels in the structure below correspond to the number labels for each resonance reported.

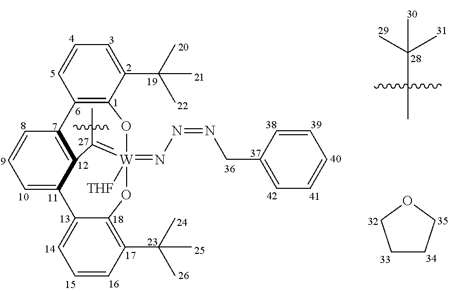

### Characterization of 1-Oct

In a vial equipped with a stir bar, complex A (200.0 mg, 0.26 mmol) was dissolved in 5 mL of benzene, then n-octyl azide (43.46 mg, 48.30 μL, 0.28 mmol) was added at room temperature. An immediate color change from dark red to light red was observed. The reaction was then stirred at room temperature for 15 min. All, volatiles were removed under vacuum yielding 1-Oct.

¹H NMR (C₆D₆, 400 MHz) δ (ppm): 7.42 (d, 2H, J=8 Hz, Ar—H_{8,10}), 7.37 (dd, 2H, J₁=2 Hz, J₂=8 Hz, Ar—H_{3,16}), 7.31 (dd, 2H, J₁=2 Hz, J₂=8 Hz, Ar—H_{5,14}), 7.24 (t, 1H, J=8 Hz, Ar—H₉), 6.85 (t, 2H, J=8 Hz, Ar—H_{4,15}), 3.72 (t, J=7 Hz, H₃₆), 3.57 (m, 4H, THF—H_{32,35}), 1.75 (m, 2H, H₃₇), 1.63 (s, 18H, Ar-^{t}Bu, H_{20-22,24-26}), 1.33 (m, 2H, H₃₈), 1.25 (m, 4H, THF—H_{33,34}), 1.23 (m, 8H, H₃₈₋₄₂), 0.90 (s, 9H, W═CC(CH₃)₃, H₂₉₋₃₁), 0.89 (m, 3H, H₄₃). The number labels in the structure below correspond to the number labels for each resonance reported.

¹³C{¹H} NMR (C₆D₆, 400 MHz) δ (ppm): 273.86 (s, W═C_{α}, C₂₇), 169.31 (s, C_{1,18}), 151.49 (s, C_{7,11}), 138.76 (s, C_{2,17}), 132.95 (s, C₉), 131.59 (s, C_{6,13}), 129.47 (s, C_{8,10}), 128.71 (s, C_{5,14}), 126.58 (s, C_{3,16}), 124.13 (s, C₁₂), 119.57 (s, C_{4,15}), 69.94 (s, C_{32,35}), 62.63 (s, C₃₆), 47.25 (s, C₂₈), 35.55 (s, C_{19,23}), 34.30 (s, C₂₉₋₃₁), 32.16 (s, C₄₁), 30.83 (s, C_{20-22,24-26}), 29.73 (s, C₄₀), 29.62 (s, C₃₉), 28.69 (s, C₃₇), 27.70 (s, C₃₈), 25.50 (s, C_{33,34}), 23.03 (s, C₄₂), 14.33 (s, C₄₃). The number labels in the structure below correspond to the number labels for each resonance reported.

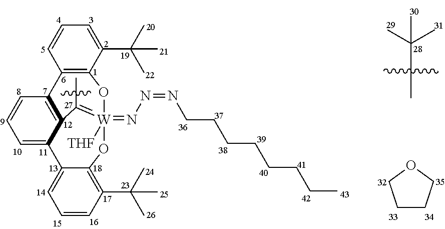

### Characterization of 1-Cy

In a vial equipped with a magnetic stir bar, complex A (100.0 mg, 0.13 mmol) was dissolved in 5 mL of benzene, then cyclohexyl azide (17.5 mg, 19.4 μL, 0.14 mmol) was added at room temperature. An immediate color change from dark red to light yellow was observed. The reaction was then stirred at room temperature for 15 min. All volatiles were removed under vacuum yielding 1-Cy.

¹H NMR (C₆D₆, 400 MHz) δ (ppm): 7.43 (d, 4H, J=7.6 Hz, Ar—H_{8,10,8′,10′}), 7.40 (dd, 4H, J₁=1.8 Hz, J₂=7.9 Hz, Ar—H_{3,16,3′,16′}), 7.33 (dd, 4H, J₁=1.8 Hz, J₂=7.9 Hz, Ar—H_{5,14,5′,14′}), 7.22 (t, 2H, J=7.6 Hz, Ar—H_{9,9′}), 6.87 (t, 4H, J=7.9 Hz, Ar—H_{4,15,4′,15′}), 3.63 (m, 2H, H_{32,32′}), 1.87 (m, 2H, Cy-H(eq.)_{33,37,33′,37′}), 1.77 (m, 2H, Cy-H(ax.)_{33,37,33′,37′}), 1.65 (s, 38H, H_{20-22,24-26,20′-22′,24′-26′}), 1.43 (m, 2H, Cy-H(ax.,eq.)_{35,35′}), 1.16 (m, 8H, Cy-H(ax.,eq.)_{34,36,34′,36′}), 0.93 (s, 18H, W═CC(CH₃)₃, H_{29-31,29-31′}). The number labels in the structure below correspond to the number labels for each resonance reported.

¹³C{¹H} NMR (C₆D₆, 400 MHz) δ (ppm): 273.57 (s, W═C_{α}, C_{27,27′}), 169.42 (s, C_{1,18,1′,18′}), 151.63 (s, C_{7,11,7′,11′}), 138.85 (s, C_{2,17,2′,17′}), 131.60 (s, C_{6,13,6′,13′}), 129.40 (s, C_{8,10,8′,10′}), 128.72 (s, C_{5,14,5′,14′}), 126.60 (s, C_{3,16,3′,16′}), 124.07 (s, C_{12,12′}), 119.51 (s, C_{4,15,4′,15′}), 69.80 (s, C_{32,32′}), 47.30 (s, C_{28,28′}), 35.58 (s, C_{19,23,19′,23′}), 34.30 (s, C_{29-31,29′-31′}), 31.68 (s, C_{33,37,33′,37′}), 30.83 (s, C_{20-22,24-26,20′-22′,24′-26′}), 25.86 (s, C_{35,35′}), 24.44 (s, C_{34,36,34′,36′}). The number labels in the structure below correspond to the number labels for each resonance reported.

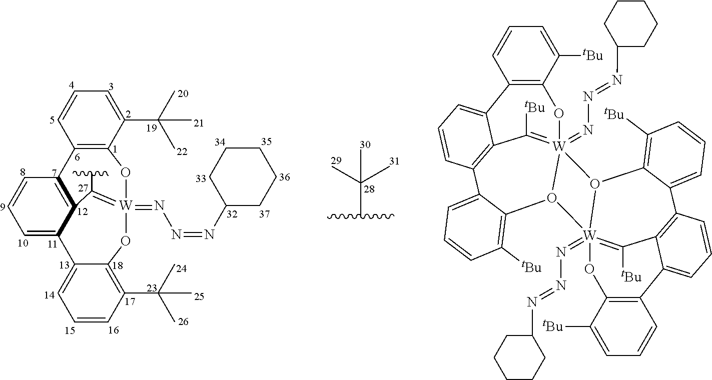

### Example 2: Synthesis of Catalyst 2

### General Procedure

In a vial equipped with a magnetic stir bar, Complex A, [^{t}BuOCO]W≡C^{t}Bu(THF)₂ (0.13 mmol) was dissolved in 5 mL of benzene. An aliquot of the appropriate azide, N₃—R (0.14 mmol) was added at room temperature. The resultant solution was then transferred in a Schlenk tube equipped with a magnetic stir bar and heated. After the appropriate reaction time, the solution was dried under vacuum yielding catalyst 2-R.

| | | R of Azide | Reaction | |
|---|---|---|---|---|
| | R group | (RN₃) | Time | Reaction Conditions |
| 2-Ph | Phenyl | Phenyl | 2 days | Benzene (6 mL); |
| | | | | 80° C. |
| 2-Bn | Benzyl | Benzyl | 3 days | Benzene (6 mL); |
| | | | | 80° C. |
| 2-TMS | Trimethylsilyl | Trimethylsilyl | 2 days | Benzene (5 mL); |
| | | | | 80° C. |
| 2-Cy | Cyclohexyl | Cyclohexyl | 3 days | Benzene (5 mL); |
| | | | | 80° C. |
| 2-H | Hydrogen | n-Octyl | 1 day | Benzene (1.5 mL); |
| | | | | 80° C. |

### Synthesis and Characterization of 2-Ph

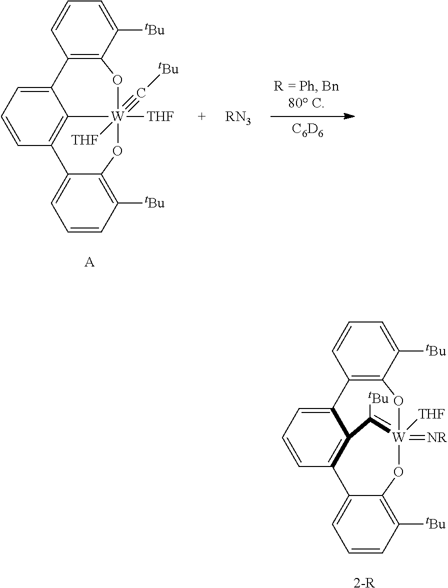

Complex A (150 mg, 0.19 mmol) was dissolved in 6 mL of benzene in a vial equipped with a magnetic stir bar, phenyl azide (25.01 mg, 23.16 μL, 0.21 mmol) was added in the above solution at room temperature. The resultant solution was then transferred in a Schlenk tube equipped with a magnetic stir bar and heated at 80° C. in oil bath for 2 days. Volatiles were removed under vacuum yielding 2-Ph.

¹H NMR (C₆D₆, 400 MHz) δ (ppm): 7.46 (m, 2H, Ar—H_{36,40}), 7.42 (d, 2H, J=6 Hz, Ar—H_{8,10}), 7.36 (m, 4H, Ar—H_{3,5,14,16}), 7.23 (t, 1H, J=6 Hz, Ar—H₉), 7.17 (m, 2H, Ar—H_{37,39}), 6.87 (m, 3H, Ar—H_{4,15,38}), 3.68 (m, 4H, THF—H_{32,35}), 1.58 (s, 18H, Ar-^{t}Bu, H_{20-22,24-26}), 1.13 (m, 4H, THF—H_{33,34}), 0.90 (s, 9H, W═CC(CH₃)₃, H₂₉₋₃₁). The number labels in the structure below correspond to the number labels for each resonance reported.

¹³C{¹H} NMR (C₆D₆, 400 MHz) δ (ppm): 272.44 (s, W═C_{α}, C₂₇), 169.22 (s, C_{1,18}), 156.76 (s, C₄₁), 150.03 (s, C_{7,11}), 138.88 (s, C_{2,17}), 132.22 (s, C₉), 132.03 (s, C_{6,13}), 129.46 (s, C_{8,10}), 128.85 (s, C_{37,39}), 128.25 (s, C_{37,39}), 126.57 (s, C₁₂), 126.42 (s, C_{3,16}), 124.05 (s, C₃₈), 119.32 (s, C_{4,15}), 72.25 (s, C_{32,35}), 46.69 (s, C₂₈), 35.44 (s, C_{19,23}), 34.21 (s, C_{29,31}), 30.84 (s, C_{20-22,24-26}), 25.36 (s, C_{33,34}). The number labels in the structure below correspond to the number labels for each resonance reported.

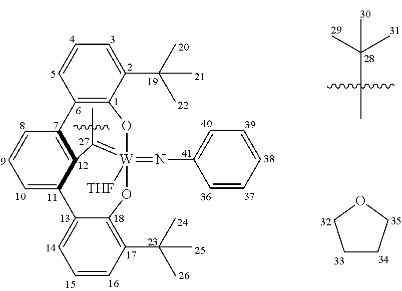

### Synthesis and Characterization of 2-Bn

In a vial equipped with a magnetic stir bar, complex A (100.0 mg, 0.13 mmol) was dissolved in 6 mL of benzene, then benzyl azide (18.64 mg, 17.5 μL, 0.14 mmol) was added at room temperature. The resultant solution was then transferred to a Schlenk tube equipped with a magnetic stir bar and heated at 80° C. in oil bath for 3d, a color change from light orange to dark brown was observed. All volatiles were removed under vacuum yielding 2-Bn.

¹H NMR (C₆D₆, 400 MHz) δ (ppm): 7.40 (m, 2H, Ar—H_{8,10}), 7.39 (m, 2H, Ar—H_{3,16}), 7.32 (dd, 2H, J₁=1.8 Hz, J₂=7.4 Hz, Ar—H_{5,14}), 7.21 (m, 1H, Ar—H₉), 7.21 (m, 2H, Ar—H_{39,41}), 7.02 (m, 1H, Ar—H₄₀), 6.88 (t, 2H, J=7.6 Hz, Ar—H_{4,16}), 6.88 (m, 2H, Ar—H_{38,42}), 5.42 (s, 2H, Bn-H₃₆), 3.52 (m, 4H, THF—H_{32,35}), 1.65 (s, 18H, Ar-^{t}Bu, H_{20-22,24-26}), 1.09 (m, 4H, THF—H_{33,34}), 0.76 (s, 9H, W═CC(CH₃)₃, H₂₉₋₃₁). The number labels in the structure below correspond to the number labels for each resonance reported.

¹³C{¹H}NMR (C₆D₆, 400 MHz) δ (ppm): 270.96 (s, W═C_{α}), 169.68 (s, C_{1,18}), 150.86 (s, C_{7,11}), 138.69 (s, C_{2,17}), 132.08 (s, C_{6,13}), 131.88 (s, C₉), 129.36 (s, C₄₀), 129.28 (s, C₃₇), 128.92 (s, C_{8,10}), 129.19 (s, C_{39,41}), 128.85 (s, C_{5,14}), 127.76 (s, C_{38,42}), 126.37 (s, C₁₂), 126.22 (s, C_{3,16}), 118.88 (s, C_{4,15}), 69.39 (s, C_{32,35}), 66.52 (s, C₃₆), 44.79 (s, C₂₈), 35.44 (s, C_{19,23}), 33.86 (s, C₂₉₋₃₁), 30.64 (s, C_{20-22,24-26}), 25.33 (s, C_{33,34}). The number labels in the structure below correspond to the number labels for each resonance reported.

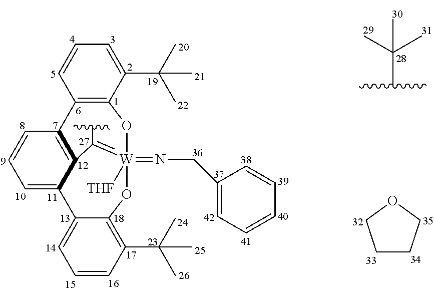

### Synthesis and Characterization of 2-Cy

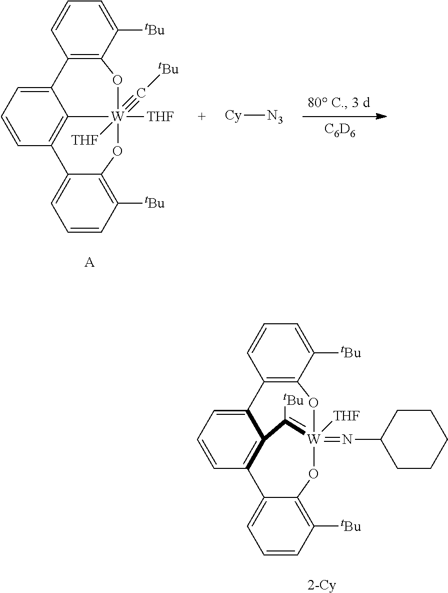

In a vial equipped with a magnetic stir bar, complex A (100.0 mg, 0.13 mmol) was dissolved in 6 mL of benzene, then cyclochexyl azide (17.5 mg, 19.4 μL, 0.14 mmol) was added at room temperature. The resultant solution was then transferred to a Schlenk tube equipped with a magnetic stir bar and heated at 80° C. in oil bath for 3d, a color change from dark red to brown-yellow was observed. All volatiles were removed under vacuum yielding 2-Cy.

¹H NMR (C₆D₆, 400 MHz) δ (ppm): 7.39 (m, 6H, Ar—H_{3,5,8,10,14,16}), 7.22 (t, 1H, J=7.5 Hz, Ar—H₉), 6.89 (2H, J=7.7 Hz, Ar—H_{4,15}), 4.39 (m, 1H, Cy-H₃₆), 3.64 (m, 4H, THF—H_{32,35}), 2.45 (d, 2H, J=10.3 Hz, Cy-H(eq.)_{37,41}), 1.70 (m, 2H, Cy-H(ax.)_{37,41}), 1.66 (s, 18H, H_{20-22,24-26}), 1.50 (d, 2H, J=11.1 Hz, Cy-H(ax.,eq.)₃₉), 1.17 (m, 4H, Cy-H(ax.,eq.)_{38,40}), 1.16 (m, 4H, THF—H_{33,34}), 0.89 (s, 9H, W═CC(CH₃)₃, H₂₉₋₃₁). The number labels in the structure below correspond to the number labels for each resonance reported.

¹³C{¹H}NMR (C₆D₆, 400 MHz) δ (ppm): 269.66 (s, W═Cₐ, C₂₇), 169.29 (s, C_{1,18}), 148.45 (s, C_{7,11}), 138.47 (s, C_{2,17}), 132.56 (s, C_{6,13}), 131.18 (s, C₉), 129.33 (s, C_{8,10}), 128.93 (s, C_{5,14}), 128.53 (s, C_{3,16}), 126.07 (s, C₁₂), 118.92 (s, C_{4,15}), 72.62 (s, C₃₆), 71.36 (s, C_{32,35}), 45.61 (s, C₂₈), 36.85 (s, C_{37,41}), 35.37 (s, C_{19,23}), 34.27 (s, C₂₉₋₃₁), 30.94 (s, C_{20-22,24-26}), 30.60 (s, C_{33,34}), 25.99 (s, C₃₉), 25.43 (s, C_{38,40}). The number labels in the structure below correspond to the number labels for each resonance reported.

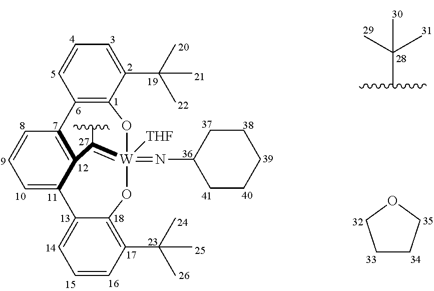

### Synthesis and Characterization of 2-TMS

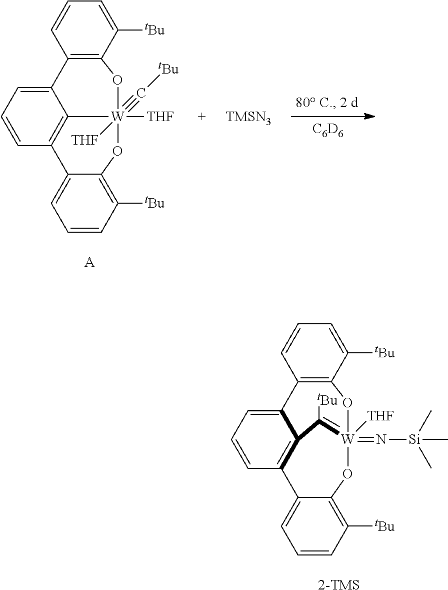

Complex A (100 mg, 0.13 mmol) was dissolved in 5 mL of benzene in a vial equipped with a magnetic stir bar. An aliquot of trimethylsilyl azide (16.4 mg, 18.8 μL, 0.14 mmol) was added at room temperature. The resultant solution was then transferred in a Schlenk tube equipped with a magnetic stir bar and heated at 80° C. in oil bath for 2 days, a color change from dark red to yellowish orange was observed. Volatiles were removed under vacuum yielding catalyst 2-TMS.

¹H NMR (C₆D₆, 500 MHz) δ (ppm): 7.37 (m, 6H, Ar—H_{3,5,8,10,14,16}), 7.19 (t, 1H, J=10 Hz, Ar—H₉), 6.89 (dd, 2H, J₁=J₂=10 Hz, Ar—H_{4,15}), 3.67 (m, 4H, THF—H_{32,35}), 1.62 (s, 18H, Ar-^{t}Bu, H_{20-22,24-26}), 1.20 (m, 4H, THF—H_{33,34}), 0.92 (s, 9H, W═CC(CH₃)₃, H₂₉₋₃₁), 0.37 (s, 9H, W═N-TMS, H₃₆₋₃₈). The number labels in the structure below correspond to the number labels for each resonance reported.

¹³C{¹H} NMR (C₆D₆, 500 MHz) δ (ppm): 272.99 (s, W═C_{α}, C₂₇), 168.56 (s, C_{1,18}), 146.93 (s, C_{7,11}), 138.42 (s, C_{2,17}), 132.38 (s, C_{6,13}), 128.84 (s, C_{5,14}), 126.33 (s, C₁₂), 126.11 (s, C_{3,16}), 119.59 (s, C_{4,15}), 72.00 (s, C_{32,35}), 44.94 (s, C₂₈), 35.51 (s, C_{19,23}), 34.36 (s, C₂₉₋₃₁), 31.26 (s, C_{20-22,24-26}), 25.48 (s, C_{33,34}), 3.45 (s, C₃₆₋₃₈). The number labels in the structure below correspond to the number labels for each resonance reported.

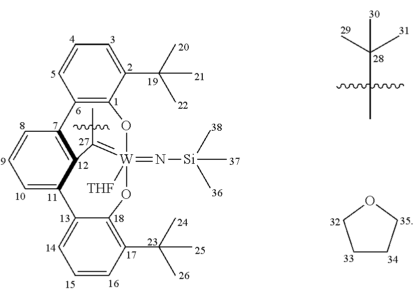

### Synthesis and Characterization of 2-H

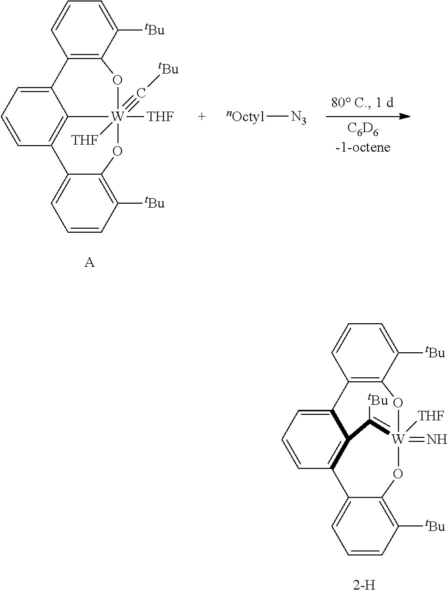

Complex A (100 mg, 0.13 mmol) was dissolved in 1.5 mL of benzene-d₆ in a vial, n-octyl azide (21.73 mg, 24.15 μL, 0.14 mmol) was added in the above solution at room temperature. The resultant solution was then transferred to a sealable NMR tube and heated at 80° C. for 3d. A color change from dark red to light red was observed as the heating was continued. The reaction mixture was allowed to cool down and the volatiles were removed under vacuum yielding 2-H.

¹H NMR (C₆D₆, 400 MHz) δ (ppm): 7.60 (bs, 1H, W═NH, H₃₆), 7.38 (m, 4H, Ar—H_{3,8,10,16}), 7.30 (dd, 2H, J₁=1 Hz, J₂=5 Hz, Ar—H_{5,14}), 7.23 (t, 1H, J=5 Hz, Ar—H₉), 6.84 (t, 2H, J=5 Hz, Ar—H_{4,15}), 5.77 (m, 1H, H₃₈), 5.00 (m, 2H, H₃₇), 3.55 (m, 8H, THF—H_{32,35}), 1.98 (m, 2H, H₃₉), 1.31 (m, 4H, H_{40,41}), 1.26 (m, 4H, THF—H_{33,34}) 1.21 (m, 8H, H_{42,43}), 0.87 (t, 3H, J=4 Hz, H₄₄), 0.74 (s, 9H, W═CC(CH₃)₃, H₂₉₋₃₁). The number labels in the structure below correspond to the number labels for each resonance reported.

¹³C{¹H} NMR (C₆D₆, 400 MHz) δ (ppm): 270.96 (s, W═C_{α}, C₂₇), 169.72 (s, C_{1,18}), 150.14 (s, C_{7,11}), 139.26 (s, C₃₈), 138.76 (s, C_{2,17}), 132.14 (s, C_{6,9,13}), 129.24 (s, C_{8,10}), 128.89 (s, C_{5,14}), 126.42 (s, C_{3,16}), 126.16 (s, C₁₂), 118.93 (s, C_{4,15}), 114.52 (s, C₃₇), 69.82 (s, C_{32,35}), 44.83 (s, C₂₈), 35.49 (s, C_{19,23}), 34.23 (s, C₃₉), 33.90 (s, C₂₉₋₃₁), 32.11 (s, C₄₂), 30.71 (s, C_{20-22,24-26}), 29.31 (s, C₄₀), 29.20 (s, C₄₁), 25.58 (s, C_{33,34}), 23.03 (s, C₄₃), 14.35 (s, C₄₄). The number labels in the structure below correspond to the number labels for each resonance reported.

¹⁵N NMR (C₆D₆, 400 MHz) δ (ppm): 337.51 (W═NH, N₃₆). The number labels in the structure below correspond to the number labels for each resonance reported.

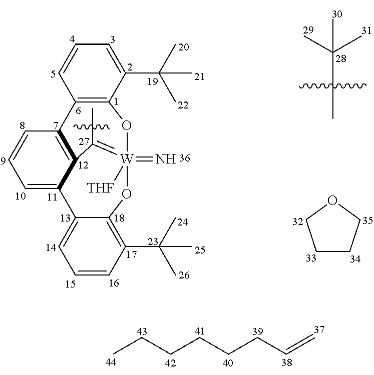

### Example 3: Polymerization of Alkenes and Alkynes with Catalyst 1

### General Procedure:

In a nitrogen atmosphere glove box, scintillation vials equipped with stir bars were charged with norbornene (100 mg, 1.06 mmol, 100 equiv) and toluene (0.1 M). To these solutions, the required amount of stock solution of catalyst 1-R (4 mg/mL) in toluene as prepared in example 1, was added in one portion with stirring. The vials were sealed and mixed. After the appropriate reaction time, the vials were removed from the glove box, unsealed and quenched with methanol. The polymers were collected via filtration and characterized with ¹H and ¹³C NMR spectroscopy.

¹H NMR of cis-syndiotactic polynorbornene (CDCl₃, 400 MHz) δ (ppm): 5.21 (m, 2H, H_{1,6}), 2.79 (m, 2H, H_{2,5}), 1.90 (dt, 1H, J₁=9.6 Hz, J₂=5.48 Hz, H₇), 1.80 (m, 2H, H_{3,4}), 1.37 (m, 2H, H_{3′,4′}), 1.01 (m, 1H, H_{7′}).

¹³C{¹H}NMR of cis-syndiotactic polynorbornene (CDCl₃, 400 MHz) δ (ppm): 134.85 (s, C_{1,6}), 43.68 (s, C₇), 39.58 (s, C_{2,5}) 34.18 (s, C_{3,4}).

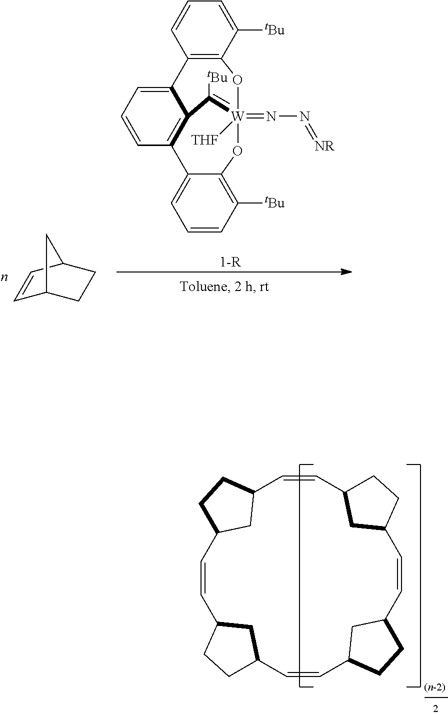

Polymerization with 1-Ph

Polynorbornene was synthesized using the general procedure outlined above with catalyst 1-Ph. The ratio of monomer:catalyst, yield, and percent cis-polynorbornene is provided in Table 1, below. ¹H and ¹³C{¹H} NMR spectra indicate that the polymer was >99% cis- and >99% syndiotactic.

Polymerization with 1-Bn

Polynorbornene was synthesized using the general procedure outlined above with catalyst 1-Bn. The ratio of monomer:catalyst, yield, and percent cis-polynorbornene is provided in Table 1, below. ¹H and ¹³C{¹H} NMR spectra indicate the polymer sample was cis-syndiotactic polynorbornene.

Polymerization with 1-Oct

Polynorbornene was synthesized using the general procedure outlined above with catalyst 1-Oct. The ratio of monomer:catalyst, yield, and percent cis-polynorbornene is provided in Table 1, below. ¹H and ¹³C{¹H} NMR spectra indicate the polymer sample was cis-syndiotactic polynorbornene.

Polymerization with 1-Cy

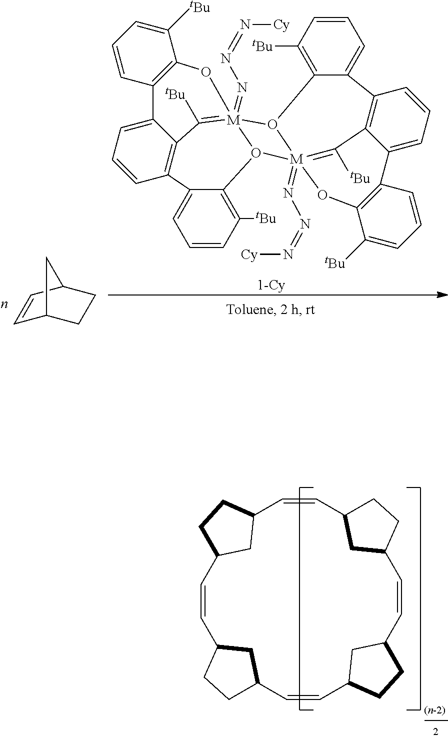

Polynorbornene was synthesized using the general procedure outlined above with catalyst 1-Cy. The ratio of monomer:catalyst, yield, and percent cis-polynorbornene is provided in Table 1, below. ¹H and ¹³C{¹H} NMR spectra indicate the polymer sample was cis-syndiotactic polynorbornene.

Thus, example 3 demonstrates general preparation conditions for preparing a cyclic polymer according to the disclosure. The catalysts of the disclosure are further characterized in example 5.

### Example 4: Polymerization of Alkenes and Alkynes with Catalyst 2

General Procedure: In a nitrogen atmosphere glove box, scintillation vials equipped with stir bars were charged with norbornene (100 mg, 1.06 mmol, 100 equiv) and toluene (0.1 M). To these solutions, the required amount of stock solution of catalyst 2-R (4 mg/mL) in toluene as prepared in Example 2, was added in one portion with stirring. The vials were sealed and mixed. After the appropriate reaction time, the vials were removed from the glove box, unsealed and quenched with methanol. The polymers were collected via filtration and characterized with ¹H and ¹³C NMR spectroscopy.

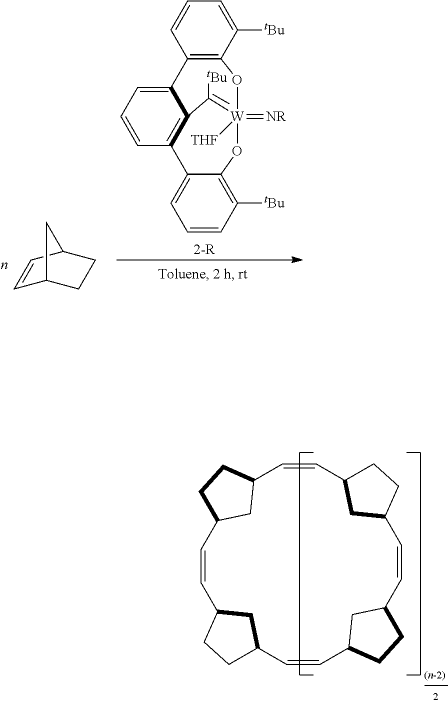

Polymerization with 2-Ph

Polynorbornene was synthesized using the general procedure outlined above with catalyst 2-Ph. The ratio of monomer:catalyst, yield, and percent cis-polynorbornene is provided in Table 1, below. ¹H and ¹³C{¹H} NMR spectra indicated the polymer sample was cis-syndiotactic polynorbornene.

Polymerization with 2-Bn

Polynorbornene was synthesized using the general procedure outlined above with catalyst 2-Bn. The ratio of monomer:catalyst, yield, and percent cis-polynorbornene is provided in Table 1, below. ¹H and ¹³C{¹H} NMR spectra indicated the polymer sample was cis-syndiotactic polynorbornene.

Polymerization with 2-TMS

Polynorbornene was synthesized using a modified procedure based on the procedure above with catalyst 2-TMS, rather than 2 h, the solution was stirred for 6 h at room temperature. The ratio of monomer:catalyst, yield, and percent cis-polynorbornene is provided in Table 1, below. ¹H and ¹³C{¹H} NMR spectra indicated the polymer sample was cis-syndiotactic polynorbornene.

Polymerization with 2-H

Polynorbornene was synthesized using a modified procedure based on the procedure above with catalyst 2-H, rather than 2 h, the solution was stirred for 6 h at room temperature. The ratio of monomer:catalyst, yield, and percent cis-polynorbornene is provided in Table 1, below. ¹H and ¹³C{¹H} NMR spectra indicated the polymer sample was cis-syndiotactic polynorbornene.

TABLE 1
Reaction times and yields for the synthesis of cyclic polynorbornene
with Catalyst 1, 1-R, and Catalyst 2, 2-R.

| | Catalyst 1-R | | | | Catalyst 2-R | | |
|---|---|---|---|---|---|---|---|
| | | Yield | | | | Yield | |
| | [M/C] | (%) | % cis^{a} | | [M/C] | (%) | % cis^{a} |
| 1-Ph | 50:1 | 99 | 99 | 2-Ph | 50:1 | 21 | 95 |
| | 100:1 | 99 | 99 | | 100:1 | 18 | 95 |
| | 200:1 | 99 | 99 | | 200:1 | 9 | 94 |
| | 500:1 | 70 | 99 | | 500:1 | — | — |
| | 1000:1 | 37 | 99 | | | | |
| 1-Bn | 50:1 | 99 | 98 | 2-Bn^{d} | 50:1 | 29 | 87 |
| | 100:1 | 99 | 99 | | 100:1 | 22 | 87 |
| | 200:1 | 97 | 97 | | 200:1 | 18 | 88 |
| | 500:1 | 65 | 99 | | 500:1 | — | — |
| | 1000:1 | 20 | 97 | | | | |
| 1-Oct | 50:1 | 99 | 99 | 2-H | 50:1 | 36 | 90 |
| | 100:1 | 99 | 99 | | 100:1 | 32 | 88 |
| | 200:1 | 95 | 99 | | 200:1 | 25 | 89 |
| | 500:1 | 79 | 99 | | 500:1 | 10 | 89 |
| | 1000:1 | 40 | 99 | | 1000:1 | — | — |
| 1-Cy | 50:1 | 85 | 99 | 2-TMS^{c} | 25:1 | 85 | 92 |
| | 100:1 | 81 | 99 | | 50:1 | 83 | 91 |
| | 200:1 | 81 | 98 | | 100:1 | 77 | 91 |
| | 500:1 | 35 | 99 | | 200:1 | 67 | 91 |
| | 1000:1 | 27 | 97 | | | | |

[M/C] = [monomer/catalyst].
^{a}Determined by ¹H NMR spectroscopy.
^{c}Reaction mixture was stirred for 6 h at room temperature.
^{d}The catalyst solution contained about 15% impurity.

Catalysts 1-R produced cyclic polymers with yields of 20% or greater and 97% or greater cis stereochemistry. Catalysts 2-R produced cyclic polymer with yields of 9% or greater and 87% or greater cis stereochemistry. Additionally, the yield decreases as the M/C ratio increases for each of catalysts 1-R and 2-R. For 1-Bn, as the M/C ratio increased from 100:1 to 1000:1, the yield fell from 99% to 20%. For 2-H, the yield fell from 36% to 10%, as the M/C ratio increased from 50:1 to 500:1. Thus, example 4 demonstrates general preparation conditions for preparing a cyclic polymer according to the disclosure. The catalysts of the disclosure are further characterized in Examples 5 and 6.

### Example 5—Polymerization of Alkenes and Alkynes with Complex B [Ru(NHC(Ad)(Mes))(═CH(PhOⁱPr))-(η²-NO₃)]

Additionally, the polymerization of norbornene was investigated with commercially-available [Ru(NHC(Ad)(Mes))(═CH(PhOⁱPr))-(η²-NO₃)] (complex B), following a literature procedure (Grubbs Et al., J. Am. Chem. Soc. 2016, 138, 1394-1405). The polymers formed from complex B, namely cis-selective (>95%) and syndiotactic (>95%) linear polynorbornene, were compared with the polymers formed using catalysts of the disclosure, cyclic polynorbornene from 1-R and 2-R.

TABLE 2
Catalysis conditions and characterization data for the synthesis of
cyclic polynorbornene according to an embodiment of the disclosure.

| | [M/C] | Mₙ^{b}(kDa) | Ð^{b} | | [M/C] | Mₙ^{b}(kDa) | Ð^{b} |
|---|---|---|---|---|---|---|---|
| 1-Ph | 50:1 | 83 | 1.99 | | 50:1 | 301 | 3.70 |
| | 100:1 | 156 | 2.42 | 2-Ph | 100:1 | 362 | 3.45 |
| | 200:1 | 118 | 1.80 | | 200:1 | 287 | 3.28 |
| | 500:1 | 303 | 1.67 | | 500:1 | — | — |
| | 1000:1 | 101 | 1.88 | | | | |
| 1-Bn | 50:1 | 194 | 2.56 | 2-Bn^{d} | 50:1 | 317 | 1.92 |
| | 100:1 | 213 | 2.17 | | 100:1 | 384 | 2.07 |
| | 200:1 | 263 | 1.73 | | 200:1 | 513 | 1.52 |
| | 500:1 | 237 | 1.89 | | 500:1 | — | — |
| | 1000:1 | 373 | 2.10 | | | | |
| 1-Oct | 50:1 | 162 | 3.18 | 2-H | 50:1 | 290 | 2.74 |
| | 100:1 | 201 | 3.09 | | 100:1 | 259 | 4.47 |
| | 200:1 | 139 | 2.81 | | 200:1 | 530 | 1.79 |
| | 500:1 | 89 | 1.95 | | 500:1 | 300 | 2.51 |
| | 1000:1 | 137 | 2.43 | | 1000:1 | — | — |
| 1-Cy | 50:1 | 195 | 3.93 | | 25:1 | 77 | 2.91 |
| | 100:1 | 196 | 3.35 | 2-TMS^{c} | 50:1 | 128 | 3.90 |
| | 200:1 | 194 | 3.02 | | 100:1 | 90 | 2.65 |
| | 500:1 | 207 | 2.85 | | 200:1 | 85 | 3.79 |
| | 1000:1 | 197 | 3.07 | | | | |

^{a}[M/C] = [monomer/catalyst].
^{b}Determined by size exclusion chromatography and multi-angle light scattering.
^{c}Reaction mixture was stirred for 6 h at room temperature.
^{d}The catalyst solution contained about 15% impurity.

### Activity and Stability of Catalysts 1-R and 2-R

The catalytic activity of catalyst 1-R was determined by quenching the reaction, as described above, after 3 min at room temperature with a monomer to catalyst ratio of 200:1. Catalysts 1-R were determined to have catalytic activities of approximately 1.00×10⁵ gₚmol⁻¹h⁻¹ (1-Ph), 8.15×10⁴ gₚmol⁻¹h⁻¹ (1-Oct), and 5.93×10⁴ gₚmol⁻¹h⁻¹(1-Bn). These activities exceed the activity of the previously reported tethered tungsten oxo, imido, and η²-bound isocyanato complexes. Moreover, the 1-R catalysts are more stable than η²-bound isocyanato complexes, which are the most active tethered tungsten catalysts known. (Gonsales, S. A. Et al., J. Am. Chem. Soc. 2016, 138 (15), 4996-4999; Jakhar, V. Et al. J. Am. Chem. Soc. 2021, 143 (2), 1235-1246; Slugovc, C. Et al. Organometallics 2005, 24 (10), 2255-2258; Bielawski, C. W. Et al. J. Am. Chem. Soc. 2003, 125 (28), 8424-8425)

The catalytic activity of the 1-R catalysts is not affected by light, which suggests that the azoimido ancillary ligand remains coordinated during polymerization and is supported by the absence of free azide in the IR spectra of polymerization reaction mixtures. Since norbornene can participate in [3+2] cycloaddition reactions with free organic azides, the effect of free azide on the polymerization rate and yield of catalyst 1-R was evaluated. (Wijnen, J. W. Et al., Tetrahedron Lett. 1995, 36, 5389-5392; Xie, S. Et, J. Am. Chem. Soc. 2015, 137, 2958-2966). An excess of azide was added to a 1:1 mixture of 1-Ph and norbornene, but excess azide did not have any noticeable effects on polymerization rate or yield, and produced no detectable cycloaddition product by ¹H NMR, suggesting the azoimdo catalyst 1-R remained intact during polymerization.

The molecular weights of polymers synthesized by azoimido catalysts 1-R are somewhat lower than the polymers synthesized by tungsten oxo and imido complexes but comparable to that of known tungsten alkylidyne complexes. (Sarkar, S. Et al., J. Am. Chem. Soc. 2012, 134, 4509-4512) While no trends for the catalysts 1-R can be readily found, it is believed the polymerization reaction produces a rapid change in viscosity during the reaction. (Jakhar, V. Et al., J. Am. Chem. Soc. 2021, 143, 1235-1246). The polymerization of norbornene exhibits slow initiation, rapid propagation, and results in formation of high molecular weight polymers that visibly alter the viscosity of the solution including precipitation of the polymer.

Additionally, initiator deactivation is believed to cause the low yields observed for imido 2-R catalysts. For example, as the M/C ratio increases, both the activity and stereoselectivity diminished for 2-Ph, producing 18% yield at a monomer/initiator ratio of 100:1 (Table 2). The low activity is in line with tungsten oxo complexes (60% yield after 7 h at room temperature). (Gonsales, S. A. Et al., J. Am. Chem. Soc. 2016, 138 (15), 4996-4999). Based on polymerization data obtained at room temperature after 3 min, the catalytic activity of 2-Ph was determined to be 487 gₚmol⁻¹h⁻¹. Based on the data presented in Table 2, a similar trend can be observed for the other imido 2-R catalysts.

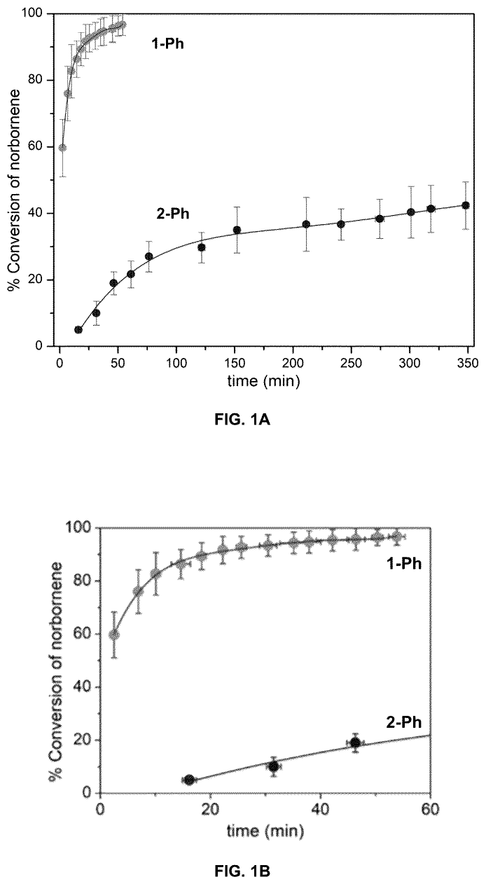

The polymerization of norbornene was evaluated in real time using catalysts 1-Ph and 2-Ph with a monomer/initiator ratio of 15:1, by collecting NMR spectra in real-time over the course of the polymerization reaction and these results are shown in FIGS. 1A and 1B. A pronounced difference in the activity of 1-Ph and 2-Ph was observed via the % conversion of norbornene (FIGS. 1A, 1B). In particular, after 60 min. the % conversion for 1-Ph was approximately 95%. After 60 min., the % conversion of 2-Ph was approximately 20%.

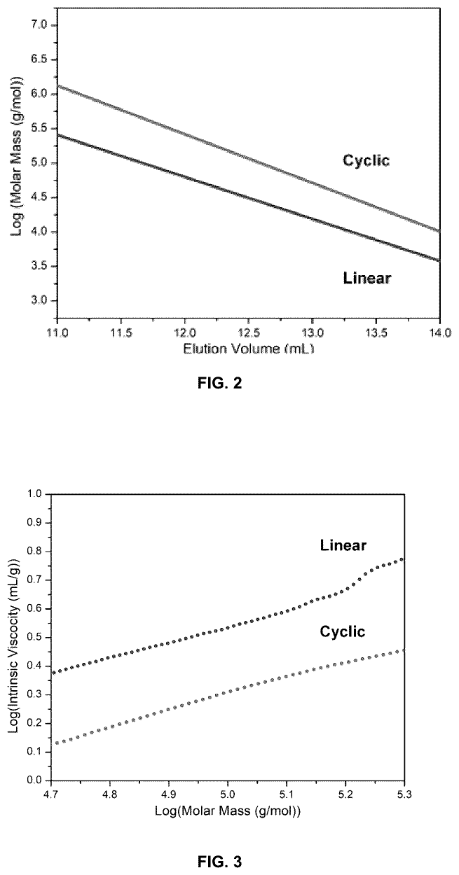

Polymer topology characterization focused on cyclic polynorbornene produced with 2-TMS. as the Mₙ (kDa) and dispersity (Ð) are most similar to those of linear polynorbornene synthesized with Grubbs' catalyst Ru(NHC(Ad)(Mes))(═CH(PhOiPr))(η2-NO₃) (Mn 79.0 kDa, Ð 2.84, >95% cis, >95% syndiotactic). Solution properties of cyclic polynorbornene were compared with those of linear norbornene via GPC analysis, providing evidence on the polymer cyclic topology. With a smaller hydrodynamic volume, cyclic polymers are expected to elute later than their linear counterparts, for a given molecular weight. As shown in the plot of log MW versus elution volume (FIG. 2), polymers prepared with catalysts of the invention generally follow this trend. Thus, the data in FIG. 2 suggests preparation of high molecular weight cyclic polymers with catalysts of the invention.

Formation of cyclic polynorbornene was confirmed by analyzing the intrinsic viscosity of the prepared polynorbornene in THF using a viscometer-equipped GPC. Due to their smaller overall dimensions, cyclic polymers are expected to exhibit lower intrinsic viscosity compared with analogous linear polymers for a given molecular weight. As shown in the Mark-Houwink-Sakurada plot in FIG. 3 where log [n] was plotted vs log M, where [n] was the intrinsic viscosity and M was the viscosity-average molar mass, the polymers prepared with the catalyst of the disclosure follow this trend.

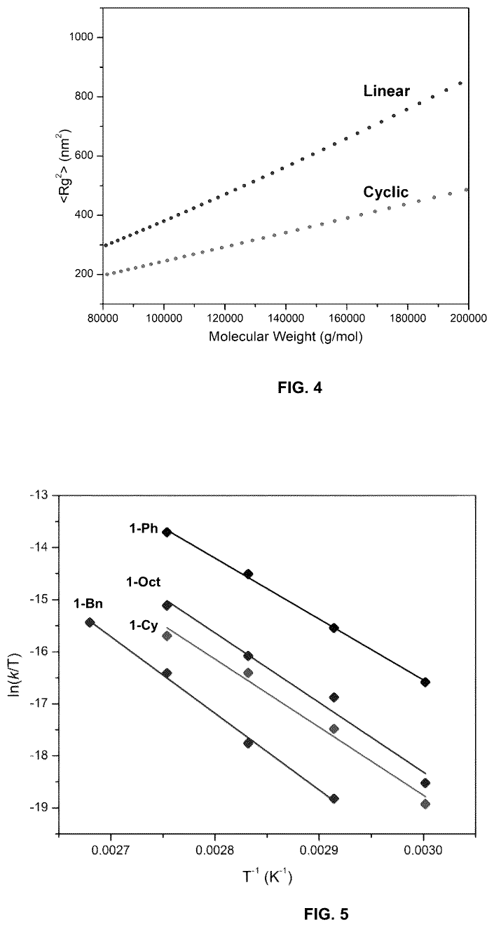

Additionally, as shown in the plot of mean square radius of gyration (<R_{g}²>) versus molar mass (FIG. 4) for the prepared polynorbornene, most of the polymers prepared with the catalysts of the disclosure exhibit lower mean square radii of gyration than the analogous polymer prepared with a catalyst not of the disclosure.

Thus, Example 5 demonstrates preparation of cyclic polymers using a catalyst of the disclosure and linear polymers using a catalyst not of the disclosure.

### Example 6—Evaluation of the Conversion of Catalysts 1-R to 2-R

Imido catalysts 2-R were observed to exhibit catalytic behavior in addition to the azoimido catalysts 1-R. The difference in activity between the 1-R and 2-R catalysts suggests the azoimido catalysts 1-R do not convert to imido catalysts 2-R under polymerization conditions. The conversion of azoimido catalyst 1-R into imido catalyst 2-R was evaluated via ¹H NMR spectroscopy and computational analysis.

The conversion of 1-R to 2-R was monitored with ¹H NMR spectroscopy to determine the rates of reaction and are shown in FIG. 5. The decay of 1-R to 2-R follows first order kinetics over a temperature range of 40° C. The increasing order of stability is 1-Ph<1-Oct<1-Cy<1-Bn, and the half-lives are 29, 116, 208, and 426 min, respectively, at 90° C. (FIG. 5). Thus, the relative stability of catalyst 1-R can vary based on the identity of R.

Additionally, an Eyring analysis was carried out on catalyst 1-R to determine the enthalpy and entropy of activation for conversion to catalyst 2-R. For catalyst 1-Ph, the enthalpy and entropy of activation were ΔH^{‡}=23.4(0.5) kcal mol⁻¹ and ΔS^{‡}=−9.8(1.4) cal mol⁻¹ K⁻¹, respectively. The negative activation entropy suggests an ordered transition state in the rate determining step. Table 3 lists the activation parameters for all 1-R.

TABLE 3
Experimental thermodynamic parameters for conversion of
1-R to 2-R calculated from Eyring equation.

| | ΔH^{‡} | ΔS^{‡} | ΔG^{‡}_{353K} |
|---|---|---|---|
| | (kcal mol⁻¹) | (cal mol⁻¹ K⁻¹) | (kcal mol⁻¹) |
| 1-Ph^{a} | 23.4 ± 0.5 | −9.8 ± 1.4 | 26.9 ± 0.7 |
| 1-Oct^{b} | 26.6 ± 2.7 | −3.9 ± 7.8 | 27.9 ± 3.9 |
| 1-Cy^{b} | 25.8 ± 2.3 | −6.6 ± 6.7 | 28.2 ± 3.3 |
| 1-Bn^{b} | 29.3 ± 1.2 | 0.6 ± 3.4 | 29.1 ± 1.7 |

^{a}Reported error bars are standard deviation calculated from three different data sets.
^{b}Reported error bars are normal uncertainty in the data obtained from single data set.

The azido→imido transformation is thermodynamically favored due to the production of N₂ gas. Consequently, the azoimido catalysts 1-R are expected to be thermodynamically unstable and have inherently low energy barriers for the transformation to the respective catalyst 2-R (Nguyen, A. I. Et al., Chem. Sci. 2011, 2, 166-189; Baek, Y. Et al., J. Am. Chem. Soc. 2020, 142, 11232-11243; Waterman, R. J. Am. Chem. Soc. 2008, 130, 12628-12629; Fickes, M. G. Et al., J. Am. Chem. Soc. 1995, 117, 6384-6385; Ison, E. A. Et al., J. Am. Chem. Soc. 2007, 129, 5, 1167-1178). Accordingly, the relative stability observed for catalyst 1-R was unexpected. (Wu, H. Et al. J. Am. Chem. Soc. 2008, 130, 16452-16453; Obenhuber, A. H. Et al., J. Am. Chem. Soc. 2014, 136, 2994-2997)

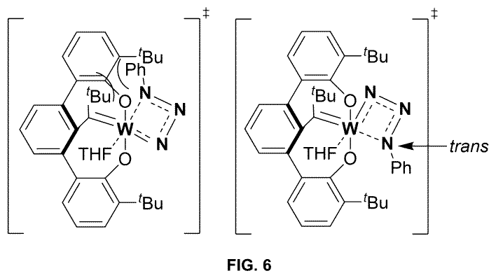

Among several postulated mechanisms, the simplest involves initial attack by the lone pair of the α-N to the W(VI) center, forming a high energy 4-centered tungstatriazeto intermediate. This unstable intermediate then expels N₂, forming a W(VI) imido via [2+2] retro-cycloaddition. In the case of 1-R, α-N attack is predicted to occur in the upper (cis to alkylidene) or lower hemisphere (trans to the alkylidene) as shown in FIG. 6. Attack cis to the alkylidene experiences steric congestion and attack trans to the alkylidene induces a negative trans effect. Furthermore, the coordinated THF is labile and can dissociate to make room for 1-R to decompose (FIG. 6).

DFT investigations were performed at the TPSSh-D0(SMD)/cc-pVTZ-(PP)//TPSSh/cc-pVDZ-(PP) level of theory to approximate the relative potential energy surface (PES) for the deazotation of catalyst 1-R. (Frisch, M. J. Et al., Gaussian 16, Revision C.01. Gaussian Inc., Wallingford CT 2016). These computational methods accurately reproduce experimental geometries and kinetic barriers of similar W-systems and match well with experimental solid-state parameters.

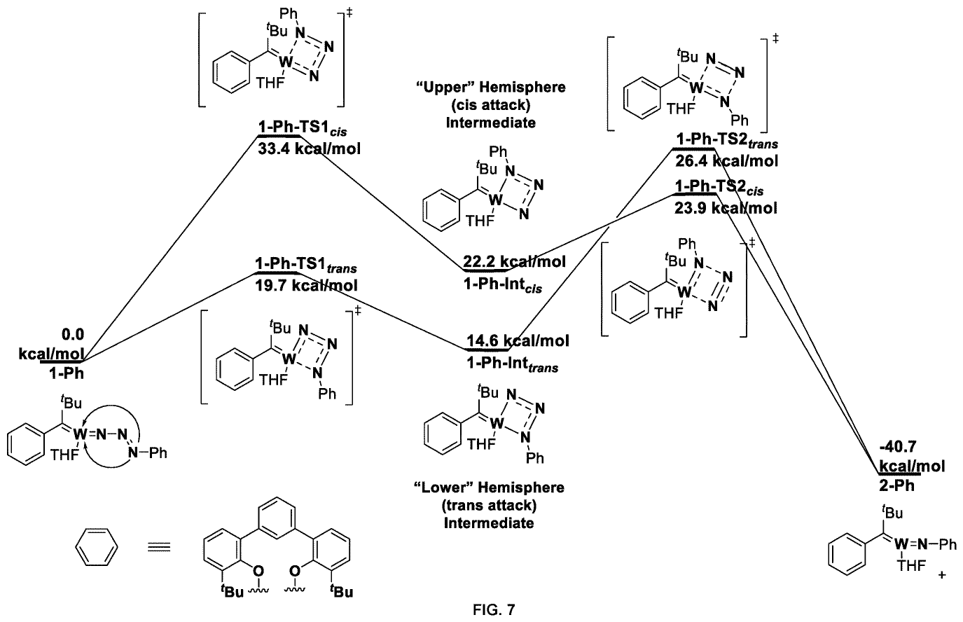

FIG. 7 depicts the potential energy surface for both the cis- (grey) and trans- (black) attack of the α-N leading to N₂ expulsion in the conversion of 1-Ph→2-Ph. The experimental results reveal an activation energy of ΔG^{‡353}=26.9±0.7 kcal mol⁻¹. The calculated barrier of ΔG^{‡353}=26.4 kcal mol⁻¹ matches the experimental kinetics. Thus, it is immediately clear the cis-attack with a predicted barrier of 33.4 kcal mol⁻¹ is too high. The high barrier is the result of a steric clash between the alkylidene tert-butyl group and the azoimido phenyl substituent. The lower barrier (2.5 kcalmol⁻¹) for [2+2] retrocycloaddition in the cis pathway is a consequence of the already elevated free energy imparted by steric congestion. The strain is still present in the intermediate 1-Ph-Int_{cis} and thus promotes easier N₂ expulsion. Thus, the trans-attack at transition state 1-Ph-TSₜᵣₐₙₛ leading to [2+2] retrocycloaddition is the lowest energy pathway. Experimentally, the near zero entropy points to an early transition state for dinitrogen expulsion. Indeed, the N—N bond remains largely intact, and the bond order changes only from 1.27 (measured by the Wiberg Bond Index) in 1-Ph-Intₜᵣₐₙₛ to 0.56 in 2-Ph-TSₜᵣₐₙₛ.

THF dissociation again offers no apparent steric relief and conversely raises the calculated transition state barriers significantly (˜10 kcal mol⁻¹). Indeed, probing the influence of sterics on the barrier height by changing the alkylidene substituent from R=^{t}Bu to R=Me lowers the barriers for cis attack more significantly (6-9 kcal mol⁻¹) than those for trans attack (1-4 kcal/mol). Accordingly, trans attack remains rate limiting.

Both attack modes require a N-atom to move into the trans-position opposite the alkylidene. However, despite the clear open space, the N-atom always avoids linearity with the alkylidene and instead experiences a ∠C═W—N˜140°. This points to a strong trans-influence from the alkylidene as the new imido bond forms. Intrinsic bond orbital (IBO) analysis at the TPPSh/def2-TZVP level of theory calculates Wiberg bond indices (WBI), indicative of relative bond strengths during the reaction. Indeed, the double bond character of the W═C_{alkylidene} bond as measured by the WBI changes during the transformation and inversely mirrors the relative energies of barriers and intermediates along the reaction pathway almost exactly. Accordingly, W—N multiple bonding in the transition state is inversely related to the strength of the alkylidene W—C bond which contributes to the azoimido thermal stability.

Thus, example 6 demonstrates that the catalysts 1-R are unexpectedly stable and that catalysts 1-R do not convert into catalysts 2-R.

Many modifications and other embodiments will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A catalyst having a structure represented by formula (I), or dimers thereof: 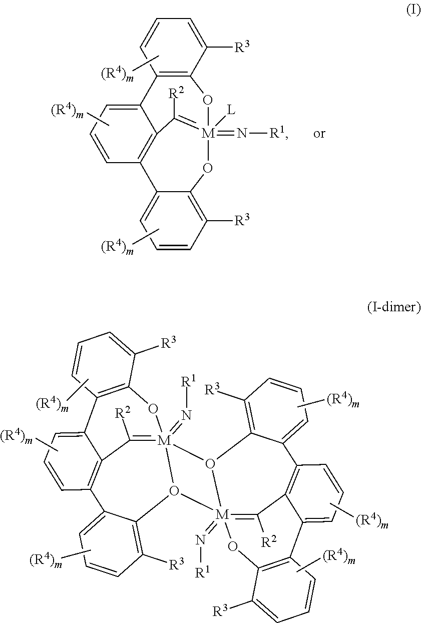
wherein
M is a transition metal;
L is absent or selected from the group of NH₃, N(R⁵)₃, Ar¹, C₁₋₆hydroxyalkyl, N≡C—R⁵, N═C(R⁵)₂, OR⁵, O(R⁵)₂, P(R⁵)₃, P(OR⁵)₃, PR⁵(OR⁵)₂, PR⁵₂(OR⁵), R⁵(CO)N(R⁵)₂, R⁵(CO)NHR⁵, R⁵CHO, R⁵COR⁵, R⁵COOR⁵, SR⁵, and S(R⁵)₂;
R¹ is selected from H, N═N—R^{a}, C₁-C₂₀alkyl, C₃₋₈cycloalkyl, C₁-C₂₀haloalkyl, Si—(R⁵)₃, and Ar¹, and R^{a} is selected from H, C₁-C₂₀alkyl, C₃₋₈cycloalkyl, and Ar¹;
R² is selected from H, Ar¹, C₁-C₂₀alkyl, C₅-C₈cycloalkyl, halo, C₁₋₂₀haloalkyl, NH₂, N(C₁-C₂₀alkyl)₂, NH(C₁-C₂₀alkyl), NHAr¹, N(Ar¹)₂, OH, OAr¹, and O(C₁-C₂₀alkyl);
each R³ is independently selected from H, Ar¹, C₁-C₂₀alkyl, C₅-C₈cycloalkyl, halo, C₁-C₂₀haloalkyl, COOH, COOC₁-C₂₀alkyl, NH₂, N(C₁-C₂₀alkyl)₂, NH(C₁-C₂₀alkyl), NHAr¹, N(Ar¹)(C₁₋₂₀alkyl), N(Ar¹)₂, SH, OAr¹, O(C₁-C₂₀alkyl), and OH, or R³ together with an adjacent R⁴ and the carbon atoms to which they are attached form a five- to eight-membered cyclic group;
each m is independently 0, 1, 2, or 3;
each R⁴ is independently selected from Ar¹, C₁-C₂₀alkyl, C₅-C₈cycloalkyl, C₁-C₂₀haloalkyl, COOH, COOC₁-C₂₀alkyl, NH₂, N(C₁-C₂₀alkyl)₂, NH(C₁-C₂₀alkyl), NHAr¹, N(Ar¹)(C₁₋₂₀alkyl), N(Ar¹)₂, SH, halo, and OH, or two adjacent R⁴, together with the carbon atoms to which they are attached, can form a five- to eight-membered cyclic group;
each R⁵ is independently selected from H, C₁-C₂₀alkyl, C₅-C₈cycloalkyl, Ar¹, or two R⁵, together with the atoms to which they are attached, form a five- to eight-membered heterocycle comprising from 1 to 3 ring heteroatoms selected from O, N, and S; and
each Ar¹ is independently selected from C₀-C₃alkylene-C₆₋₂₀ aryl, or a 5-12 membered heteroaryl comprising from 1 to 3 ring heteroatoms selected from O, N, and S.

2. The catalyst of claim 1, wherein M comprises a group 6 transition metal.

3. The catalyst of claim 2, wherein M is chromium (Cr), molybdenum (Mo), or tungsten (W).

4. (canceled)

5. The catalyst of claim 1, wherein R² is H, Ar¹, C₁-C₂₀ alkyl, C₅-C₈cycloalkyl, or C₁-C₂₀haloalkyl.

6. (canceled)

7. The catalyst of claim 5, wherein R² is tert-butyl.

8. The catalyst of claim 1, wherein each R³ is independently selected from H, Ar¹, C₁-C₂₀alkyl, C₅-C₈cycloalkyl, halo, C₁-C₂₀haloalkyl, COOH, COOC₁-C₂₀alkyl, NH₃, SH, and OH.

9. The catalyst of claim 8, wherein each R³ is independently H, Ar¹, C₁-C₂₂ haloalkyl or C₁-C₂₀alkyl.

10. The catalyst of claim 1, wherein R³ is tert-butyl, C₆₋₂₀aryl, or C₁₋₃alkylene-C₆₋₂₀aryl.

11. (canceled)

12. The catalyst of claim 1, wherein each R⁴ is independently selected from C₁-C₂₀alkyl, C₅-C₈cycloalkyl, Ar¹, COOH, COOC₁-C₂₀alkyl, NH₃, SH, halo, C₁-C₂₀haloalkyl, and OH.

13. The catalyst of claim 12, wherein each R⁴ is C₁-C₂₀alkyl, C₅-C₈cycloalkyl, C₁-C₂₀haloalkyl, C₆₋₂₀aryl, or C₁₋₃ alkylene-C₅₋₂₀ aryl.

14.-15. (canceled)

16. The catalyst of claim 1, wherein L is Ar¹, N(R⁵)₃, P(R⁵)₃, S(R⁵)₂ or O(R⁵)₂.

17. The catalyst of claim 16, wherein each R⁵ of L is independently selected from C₁-C₂₀alkyl, C₅-C₈cycloalkyl, and Ar¹C₆₋₂₀aryl, and C₁₋₃alkyleneC₆₋₂₀ aryl.

18. (canceled)

19. The catalyst of claim 1, wherein L is tetrahydrofuran (THF), pyridine, or thiophene.

20. The catalyst of claim 1, wherein R¹ is selected from the group consisting of H, C₁₋₂₀alkyl, C₃₋₈cycloalkyl, and Ar¹.

21. The catalyst of claim 1, wherein R¹ is H, N═N—R^{a}, C₁₋₂₀alkyl, C₃₋₈ cycloalkyl, or Ar¹.

22. The catalyst of claim 21, wherein each Ar¹ of R¹ is C₀-C₃alkylene-C₆₋₂₀ aryl.

23. The catalyst of claim 1, wherein R¹ is phenyl, C₈alkyl, C₆cycloalkyl, or benzyl.

24. The catalyst of claim 21, wherein R¹ is N═N—R^{a}, and R^{a} is C₁-C₂₂ alkyl, C₃₋₈ cycloalkyl, C₆₋₂₀aryl, or C₁₋₃alkylene-C₆₋₂₀aryl.

25.-26. (canceled)

27. A method for making the catalyst of claim 1, comprising
reacting a compound of formula (II) and an organoazide having a structure of formula (III) under conditions sufficient to form the catalyst: 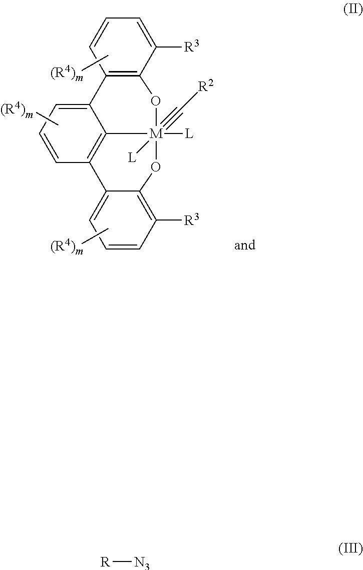
wherein in formula (II):
M is a transition metal;
each L is independently absent or selected from the group of NH₃, N(R⁵)₃, Ar¹, C₁₋₆hydroxyalkyl, N≡C—R⁵, N═C(R⁵)₂, OR⁵, O(R⁵)₂, P(R⁵)₃, P(OR⁵)₃, PR⁵(OR⁵)₂, PR⁵₂(OR⁵), R⁵(CO)N(R⁵)₂, R⁵(CO)NHR⁵, R⁵CHO, R⁵COR⁵, R⁵COOR⁵, SR⁵, and S(R⁵)₂;
R² is selected from H, Ar¹, C₁₋₂₀alkyl, C₅-C₈cycloalkyl, halo, C₁₋₂₀haloalkyl, NH₂, N(C₁₋₂₂ alkyl)₂, NH(C₁₋₂₂ alkyl), NHAr¹, N(Ar¹)(C₁₋₂₀alkyl), N(Ar¹)₂, OH, OAr¹, O(C₁₋₂₂ alkyl), and Si—(R⁵)₃;
each R³ is independently selected from H, Ar¹, C₁-C₂₀alkyl, C₅-C₈cycloalkyl, halo, C₁-C₂₀haloalkyl, COOH, COOC₁₋₂₀alkyl, NH₂, N(C₁₋₂₀alkyl)₂, NH(C₁₋₂₀alkyl), NHAr¹, N(Ar¹)(C₁₋₂₀alkyl), N(Ar¹)₂, SH, OAr¹, O(C₁₋₂₀alkyl), and OH, or R³ together with an adjacent R⁴ and the carbon atoms to which they are attached form a five- to eight-membered cyclic group;
each R⁴ is independently selected from Ar¹, C₁-C₂₀alkyl, C₅-C₈cycloalkyl, C₁-C₂₀haloalkyl, COOH, COOC₁₋₂₀alkyl, NH₂, N(C₁₋₂₀alkyl)₂, NH(C₁₋₂₀alkyl), NHAr¹, N(Ar¹)(C₁₋₂₀alkyl), N(Ar¹)₂, SH, halo, and OH, or two adjacent R⁴, together with the carbon atoms to which they are attached, can form a five- to eight-membered cyclic group;
each R⁵ is independently selected from H, C₁-C₂₀alkyl, C₅-C₈cycloalkyl, Ar¹, or two R⁵, together with the atoms to which they are attached, form a five- to eight-membered heterocycle comprising from 1 to 3 ring heteroatoms selected from O, N, and S; and
each Ar¹ is independently selected from C₆₋₂₀aryl, C₁₋₃alkylene-C₆₋₂₀aryl, or a 5-12 membered heteroaryl comprising from 1 to 3 ring heteroatoms selected from O, N, and S; and
each m is independently 0, 1, 2, or 3; and
wherein in formula (III):
R is selected from H, C₁₋₂₀alkyl, C₃₋₈cycloalkyl, Si—(R⁵)₃, and Ar¹.

28.-34. (canceled)

35. A method of preparing a cyclic polymer, comprising:
admixing a plurality of alkene monomers, alkyne monomers, or both in the presence of the catalyst of formula (I), or dimer thereof, of claim 1 under conditions sufficient to polymerize the plurality of alkene monomers, alkyne monomers, or both to form the cyclic polymer, wherein the cyclic polymer ring comprises alkene groups.

36.-44. (canceled)
